(19) United States
(12) Patent Application Publication
(10) Pub. No.: US 2010/0086997 A1
(43) Pub. Date: Apr. 8, 2010

(54) TrkB Agonists for Treating Autoimmune Disorders

(75) Inventors: Chia-Yang Lin, Palo Alto, CA (US); Hua Long, Stanford, CA (US); David Liang Tsao, Cupertino, CA (US)

Correspondence Address: PFIZER INC, 10555 SCIENCE CENTER DRIVE, SAN DIEGO, CA, 92121, US

(73) Assignee: RINAT ENUROSCIENCE CORP

(21) Appl. No.: 12/519,743

(22) PCT Filed: Dec. 5, 2007
(86) PCT No.: PCT/IB2007/004145

§ 371 Date: Jun. 17, 2009

Related U.S. Application Data
(60) Provisional application No. 60/870,918, filed on Dec. 20, 2006.

(51) Int. Cl. C12N 5/12; C07K 14/475; C07K 16/00

(52) U.S. Cl. 435/346; 530/350; 530/387.1; 530/388.1

(57) ABSTRACT

Tyrosine receptor kinase (TrkB) agonists are provided for reducing leukocyte invasion of the central nervous system in autoimmune diseases such as multiple sclerosis. TrkB agonists include naturally-occurring agonists, such as NT4 and BDNF, as well as agonists such as agonist antibodies.

This application claims priority to U.S. Provisional Application No. 60/870,918 filed on Dec. 20, 2006, which is incorporated herein by reference in its entirety.

### FIELD OF THE INVENTION

The invention relates to the treatment of autoimmune diseases affecting the central nervous system, including multiple sclerosis. The invention provides tyrosine receptor kinase B (TrkB) agonists for reducing leukocyte invasion of central nervous system tissues.

### BACKGROUND

Multiple sclerosis (MS) is a demyelinating autoimmune disease of the central nervous system (CNS) characterized by inflammation, demyelination, and axonal injury. The disease affects more than a million people world-wide and is twice as prevalent in women than in men. The symptoms of MS usually appear between age 20 and 40.

The etiology of MS is unclear, although features of the disease have been studied. Such features include damage to CNS tissues, activation of microglia, proinflammatory cytokine production, arrest of T-cell migration and clonotypic expansion, altered macrophage effector function, production, upregulation of MHC, and direct CNS attack by infiltrating T-cells.

Experimental autoimmune encephalomyelitis (EAE) is considered a standard model for MS. The murine disease model has been used to study the etiology of MS and to evaluate drugs for its treatment (Aharoni, R. et al., 2005a). The clinical features of EAE include inflammation and demyelination of the CNS by large numbers of infiltrating lymphocytes and macrophages. Active immunization of mice with several different protein components of myelin, including myelin basic protein (MBP), proteolipid protein (PLP), and myelin oligodendrocyte glycoprotein (MOG), induces the production of autoimmune antibodies and the clinical symptoms of ascending paralysis. The disease may be acute or chronic depending on the mouse strain and the myelin protein used for immunization. EAE has been used to study the etiology of MS and to evaluate drugs for its treatment (Aharoni, R. et al., 2005a).

MS results largely from a T-cell response to myelin, a polypeptide abundant in neuronal tissues. Demyelination and clinical paralysis result from the invasion of CNS tissues by T-cells of the Th1 phenotype, with specificity for myelin antigens. Th1 cells produce inflammatory cytokines including TNF-α and IFN-γ. Damage to the CNS is likely also caused by other immunological responses, including the production of autoimmune antibodies and complement activation. B-cells are involved during early and late stages of MS and EAE with myelin basic protein (MBP)-specific antibodies of various isotypes found throughout the courses of the diseases. Sections prepared from brain and spinal cord tissues show leukocyte invasion (particularly lymphocytes and macrophages) and destruction of the underlying tissues of the nervous system).

Glatiramer acetate (GA, COPAXONE) is an immunosuppressive drug approved for the treatment of MS and other diseases (Arnon, R. et al., 2003). The drug is also effective in the EAE model. GA is an inducer of Th2/3 cells, which produce anti-inflammatory cytokines, which cross the blood-brain barrier to accumulate in the CNS. These cytokines produce various effects in tissue of the CNS, and lead to the local production of other growth factors, e.g., IL-10, TGF-β, and BDNF (Aharoni, R. et al., 2003). Prolonged GA administration was associated with higher levels of expression of the NT3, NT4, and BDNF (Aharoni, R. et al., 2005b).

NT3, NT4, and BDNF are members of the neurotrophin (NT) family of small homodimeric proteins important for neuronal development, process growth, synaptic plasticity, protection, and survival. NTs include nerve growth factor (NGF), brain-derived neurotrophic factor (BDNF), NT3, NT4 (also called NT4/5), NT6, and NT7. NTs affect target cells through interactions with a family of receptors, known as receptor tyrosine kinases (also called tyrosine receptor kinases). These receptors include several high molecular weight (130-150 kDa), high-affinity (˜10⁻¹¹ M) tyrosine receptor kinase (Trk) receptors and a receptor known as low molecular weight (65-80 kDa), low affinity (˜10⁻⁹ M) receptor (LNGFR, p75NTR, or p75). NT binding to a specific receptor tyrosine kinase causes receptor dimerization and activation of the intrinsic tyrosine kinase domain. NGF binds preferentially to tyrosine receptor kinase A (TrkA), BDNF and NT4 to tyrosine receptor kinase B (TrkB), and NT3 to tyrosine receptor kinase C (TrkC). All NTs bind weakly to p75.

The report of BDNF and NT4 in the CNS tissues of EAE mice (Aharoni, R. et al., 2003, 2005b) suggests a role for NTs and/or their receptors in multiple sclerosis and related diseases.

### REFERENCES

Aharoni, R. et al. (2005a) J. Neurosci. 25:8217-28.
Aharoni, R. et al. (2005b) Proc. Nat'l. Acad. Sci. USA 102:19045-50.
Aharoni, R. et al. (2003) Proc. Nat'l. Acad. Sci. USA 100:14157-62.
Aharoni, R. et al. (1997) Proc. Nat'l. Acad. Sci. USA 94:10821-26.
Arnon, R. et al. (2003) J. Mol. Recognit. 16:412-21.
Davies, A. et al. (1993) Neuron 11565-74.
Karnezis, T. et al. (2004) Nat. Neurosci. 7:736-44.
Padlan, E. et al., (1995) FASEB J. 133-39.
Steinman and Zamvil (2006) Ann. Neurol. 60:12-21.

Additional references, particularly references relating to standard procedures and methods, are cited throughout the text. All patents, patent applications, Genbank entries, reference manuals, and publications cited above and elsewhere in the application are hereby incorporated by reference in their entirety.

### BRIEF SUMMARY OF THE INVENTION

In one aspect, the invention provides a method for reducing leukocyte invasion of a tissue of the central nervous system comprising administering to a mammalian subject in need of such treatment a composition comprising a TrkB agonist in an amount effective for activating the TrkB receptor, thereby reducing leukocyte invasion of a tissue of the central nervous system. The present invention further provides the use of a TrkB agonist in the manufacture of a medicament used for reducing leukocyte invasion of a tissue of the central nervous system in a mammal.

In some embodiments, the mammal has an autoimmune disorder. In one embodiment, the autoimmune disorder is experimental autoimmune encephalomyelitis. In another embodiment, the autoimmune disorder is multiple sclerosis. In other embodiments, the autoimmune disease is associated with immune rejection, optic neuropathies, inflammatory bowel disease, or Parkinson's disease. In preferred embodiments, the mammal is a human.

In some embodiments, the TrkB agonist is a naturally-occurring TrkB agonist. In one embodiment, the naturally-occurring TrkB agonist is NT4. In related embodiments, the naturally-occurring TrkB agonist comprises a naturally-occurring fragment or derivative of NT4. In some embodiments, the fragment or derivative of NT4 binds to and activates TrkB. In another embodiment, the naturally-occurring TrkB agonist is BDNF. In related embodiments, the naturally-occurring TrkB agonist comprises a naturally-occurring fragment or derivative of BDNF. In some embodiments, the fragment or derivative of BDNF binds to and activates TrkB.

In another embodiment, the TrkB agonist is an antibody. In a particular embodiment, the antibody is antibody 38B8. In another embodiment, the antibody is produced by the hybridoma strain deposited under ATCC Deposit Number PTA-8766. In some embodiments, the TrkB agonist is a human antibody. In related embodiments, the TrkB agonist is a humanized antibody. In some embodiments the TrkB agonist is an antibody fragment or derivative. In particular embodiments, the antibody fragment is selected from an Fab, Fab′, F(ab′)2, Fv, Fc, or single chain (ScFv) antibody. In some embodiments the antibody fragment comprises the antigen-binding region of agonist antibody 38B8. In some embodiments the antibody fragment comprises the antigen-binding region of the antibody produced by the hybridoma strain deposited under ATCC Deposit Number PTA-8766. In a related embodiment, the antibody fragment comprises the complementarity determining regions (CDR) of agonist antibody 38B8. In a related to embodiment, the antibody fragment comprises the complementarity determining regions (CDR) of the antibody produced by the hybridoma strain deposited under ATCC Deposit Number PTA-8766.

In preferred embodiments, the invading leukocytes comprise T-cells and macrophages. In particular embodiments, the invading leukocytes comprise CD3-expressing and CD68-expressing leukocytes. In preferred embodiments, the tissue of the central nervous system is brain tissue or spinal cord tissue.

In a related aspect, the invention provides a method for treating an autoimmune disorder affecting the central nervous system comprising administering to a mammalian subject in need of such treatment a composition comprising an amount of a TrkB agonist sufficient to activate the TrkB receptor, thereby reducing leukocyte invasion of tissues of the central nervous system. The present invention further provides the use of a TrkB agonist in the manufacture of a medicament used for treating an autoimmune disorder affecting the central nervous system in a mammal.

In one embodiment, the autoimmune disorder is experimental autoimmune encephalomyelitis. In another embodiment, the autoimmune disorder is multiple sclerosis. In other embodiments, the autoimmune disease is associated with immune rejection, optic neuropathies, inflammatory bowel disease, or Parkinson's disease. In preferred embodiments, the mammal is a human.

In some embodiments, the TrkB agonist is a naturally-occurring TrkB agonist. In one embodiment, the naturally-occurring TrkB agonist is NT4. In related embodiments, the naturally-occurring TrkB agonist comprises a naturally-occurring fragment or derivative of NT4. In some embodiments, the fragment or derivative of NT4 binds to and activates TrkB. In another embodiment, the naturally-occurring TrkB agonist is BDNF. In related embodiments, the naturally-occurring TrkB agonist comprises is a naturally-occurring fragment or derivative of BDNF. In some embodiments, the fragment or derivative of BDNF binds to and activates TrkB.

In another embodiment, the TrkB agonist is an antibody. In a particular embodiment, the antibody is antibody 38B8. In another embodiment, the antibody is produced by the hybridoma strain deposited under ATCC Deposit Number PTA-8766. In some embodiments, the TrkB agonist is a human antibody. In related embodiments, the TrkB agonist is a humanized antibody. In some embodiments the TrkB agonist is an antibody fragment or derivative. In particular embodiments, the antibody fragment is selected from an Fab, Fab′, F(ab′)2, Fv, Fc, or single chain (ScFv) antibody. In some embodiments the antibody fragment comprises the antigen-binding region of agonist antibody 38B8. In some embodiments the antibody fragment comprises the antigen-binding region of the antibody produced by the hybridoma strain deposited under ATCC Deposit Number PTA-8766. In a related embodiment, the antibody fragment comprises the complementarity determining regions of agonist antibody 38B8. In a related to embodiment, the antibody fragment comprises the complementarity determining regions (CDR) of the antibody produced by the hybridoma strain deposited under ATCC Deposit Number PTA-8766.

In preferred embodiments, the invading leukocytes comprise T-cells and macrophages. In particular embodiments, the invading leukocytes comprise CD3-expressing and CD68-expressing leukocytes. In preferred embodiments, the tissue of the central nervous system is brain tissue or spinal cord tissue.

In a further aspect, the invention provides a kit of parts for reducing leukocyte invasion of a tissue of the central nervous system, comprising a TrkB agonist in an amount effective for activating the TrkB receptor and instructions for use. In a related aspect, the invention provides a kit of parts for treating an autoimmune disorder affecting the central nervous system, comprising a TrkB agonist in an amount effective for activating the TrkB receptor and instructions for use.

In a further embodiment, the present invention relates to the TrkB agonist antibody 38B8, for example an isolated monoclonal 38B8 antibody. In another embodiment, the present invention relates to an isolated TrkB agonist antibody produced by the hybridoma strain deposited under ATCC Deposit Number PTA-8766. In some embodiments the TrkB agonist is an antibody fragment or derivative of 38B8. In a further embodiment, the TrkB agonist is an antibody fragment or derivative of the antibody produced by the hybridoma strain deposited under ATCC Deposit Number PTA-8766. In particular embodiments, the antibody fragment is selected from an Fab, Fab′, F(ab′)2, Fv, Fc, or single chain (ScFv) antibody derived from 38B8. In particular embodiments, the antibody fragment is selected from an Fab, Fab′, F(ab′)2, Fv, Fc, or single chain (ScFv) antibody derived from the antibody produced by the hybridoma strain deposited under ATCC Deposit Number PTA-8766. In some embodiments the antibody fragment comprises the antigen-binding region of agonist antibody 38B8. In some embodiments the antibody fragment comprises the antigen-binding region of the agonist antibody produced by the hybridoma strain deposited under ATCC Deposit Number PTA-8766. In a related embodiment, the antibody fragment comprises the complementarity determining regions (CDR) of agonist antibody 38B8. In a related embodiment, the antibody fragment comprises the complementarity determining regions (CDR) of agonist antibody that is produced by the hybridoma strain deposited under ATCC Deposit Number PTA-8766. In a further embodiment is a cell that produces the TrkB agonist antibody 38B8, or that produces a fragment derived from 38B8. In a further embodiment is the hybridoma strain deposited under ATCC Deposit Number PTA-8766.

The present invention also relates to any of the TrkB agonists described herein for use in treating any of the autoimmune disorders described herein. The present invention also provides a pharmaceutical composition comprising any of the TrkB agonists as described herein and a pharmaceutically acceptable carrier.

In another embodiment, the invention relates to an isolated nucleic acid molecule comprising a nucleotide sequence that encodes any of the TrkB agonists as described herein. In one particular embodiment is an isolated nucleic acid molecule comprising a nucleotide sequence that encodes the agonist antibody that is produced by the hybridoma strain deposited under ATCC Deposit Number PTA-8766. The invention further relates to a vector comprising any of the nucleic acid molecules described herein, wherein the vector optionally comprises an expression control sequence operably linked to the nucleic acid molecule.

Another embodiment provides a host cell comprising any of the vectors described herein or comprising any of the nucleic acid molecules described herein. The present invention also provides an isolated cell line that produces any of the antibodies or antigen-binding portions as described herein or that produces the heavy chain or light chain of any of said antibodies or said antigen-binding portions.

In another embodiment, the present invention relates to a method for producing a TrkB agonist antibody or antigen-binding portion thereof, comprising culturing any of the host cells or cell lines described herein under suitable conditions and recovering said antibody or antigen-binding portion.

The present invention also relates to a non-human transgenic animal or transgenic plant comprising any of the nucleic acids described herein, wherein the non-human transgenic animal or transgenic plant expresses said nucleic acid.

The present invention further provides a method for isolating a TrkB agonist antibody or antigen-binding portion thereof, comprising the step of isolating the antibody from the non-human transgenic animal or transgenic plant as described herein.

These and other aspects of the invention will become apparent from the description of the invention and examples that follow.

### BRIEF DESCRIPTION OF THE DRAWINGS

FIGS. 1A and 1B depict graphs showing the relative morbidity in EAE animals in the weeks following MOG-induction. MOG was administered on day 0. (A) Beginning at day 10 animals received daily administrations of either NT4 (10 mg/kg) or vehicle alone as a control. (B) Animals were treated with either control IgG (10 mg/kg; n=9), NT4 (10 mg/kg) from day 3 to day 9 (n=8), or NT4 (10 mg/kg) from day 9 to day 15 (n=8).

FIG. 2 depicts a series of graphs showing the relative affinity of four receptor tyrosine kinase agonists (i.e., NGF, BDNF, NT4, and 38B8) for four receptor tyrosine kinases (i.e., TrkA, TrkB, TrkC, and p75). The x-axis shows time (all in the range of 300-450 seconds). The y-axis shows relative affinity. Full scale top row (left to right): 90, 20, 50 and 60 units; second row: 32, 60, 60, 140 units; third row: 35, 35, 16, and 20 units; bottom row: 90, 80, 100, and 90 units. These data are further described in the text and in Example 4 (including Table 2).

FIG. 3 depicts a graph showing morbidity in animals treated with a TrkB agonist 9 days following MOG-induction. Animals received on days 9 and 16 either 5 mg/kg TrkB agonist antibody 38B8 or a non-specific IgG (control). Mice were assessed daily for clinical signs of EAE according to the following scoring system: 0=normal; 1=limp tail; 2=moderate hind-limb weakness; 3=moderately severe hind-limb weakness (animal can still walk with difficulty); 4=severe hind-limb weakness (animal can move their hind-limbs but cannot walk); 5=complete hind limb paralysis; and 6=death.

FIG. 4 depicts a graph showing morbidity in animals treated with a TrkB agonist 16 days following MOG-induction. Animals received on days 16 and 23 either 5 mg/kg TrkB agonist antibody 38B8 or vehicle (PBS).

FIGS. 5A and 5B depict graphs showing morbidity in animals treated later in disease progression. (A) Animals received on days 18 and 23 either 5 mg/kg TrkB agonist antibody 38B8 or a non-specific IgG. (B) Animals received beginning on day 22 daily administrations of either GA (COPAXONE) or vehicle only (control).

FIGS. 6A and 6B depict graphs showing morbidity and body weight in animals following the administration of either a TrkB agonist or dexamethasone. Animals received either 38B8 (5 mg/kg, weekly on day 9 and day 16), or dexamethasone (4 mg/kg) or ethanol vehicle (control), daily on days 3-12. (A) Morbidity was measured as above. (B) The body weight of the animals was measured over the course of disease progression.

FIGS. 7A and 7B depict graphs showing that the effectiveness of the TrkB agonist in reducing disease morbidity is dosage-dependent. (A) Animals received on days 9 and 16 either 1 mg/kg or 5 mg/kg of the TrkB agonist 38B8. (B) Animals received on days 9 and 16 either 5 mg/kg 38B8 or 10 mg/kg 38B8, or PBS (control).

FIG. 8 depicts a graph showing the relative amount of neuron survival in the presence of increasing amounts of several TrkB agonist antibodies (38B8, 23B8, 36D1, 37D12, and 19H8) using an in vitro assay. The experimental procedures and results are described in the text and in Example 1 (e.g., Table 1). The EC₅₀ value for each antibody in the neuron survival assay is shown in the 3^{rd} column of Table 1.

FIG. 9 depicts a series of graphs showing the relative levels of the indicated isotypes of MOG-specific antibodies in untreated (control) animals and animals treated with the TrkB agonist antibody 38B8. Each graph shows the amount of the indicated antibody isotype (as determined by measuring absorption at 450 μm) in TrkB agonist (38B8)-treated or untreated (control) animals.

FIG. 10 depicts a graph showing the results of a splenocyte stimulation assay. Spleen cells from MOG-induced untreated (control) animals or MOG-induced TrkB agonist antibody (38B8)-treated animals were cultured in vitro in the presence of MOG alone or MOG in combination with TrkB agonist antibody (38B8; 50 μl/mg), or dexamethasone at one of two different concentrations (10⁻⁸ M and 10⁻⁵ M).

FIG. 11 depicts the results of immunochemical staining of spinal cord sections removed from control (A) and TrkB agonist-treated animals (B). The sections were stained with Luxol Fast Blue for myelin and Cresyl violet for cell bodies.

FIG. 12 depicts the results of immunochemical staining of spinal cord sections removed from control (A) and TrkB agonist-treated animals (B). The sections were stained with an antibody specific for CD3.

FIG. 13 depicts the results of immunochemical staining of spinal cord sections removed from control (A) and TrkB agonist-treated EAE animals (B). The sections were stained with an antibody specific for CD68.

FIG. 14 depicts the results of histological staining of spinal cord sections removed from control and TrkB agonist-treated EAE animals. The sections were stained with a myelin stain, Luxol Fast Blue. The absence of staining indicates areas of demyelination.

### DETAILED DESCRIPTION

### Definitions

As used herein, the terms “tyrosine receptor kinase” and “receptor tyrosine kinase” are used interchangeably to refer to a class of molecules of which TrkB is a member. The binding of a ligand (agonist) to a receptor tyrosine kinase triggers ligand-induced receptor dimerization and autophosphorylation of tyrosine residues in the intracellular kinase domain. Tyrosine phosphorylation is followed by the activation of diverse signaling cascades, such as the phosphatidylinositol 3-kinase (PI3K)/Akt, MAPK, and PLC-pathways, which modulate gene expression, typically in a cell type-specific manner.

As used herein, “invading leukocytes” are leukocytes that invade, infiltrate, or migrate into tissues of the central nervous system (CNS), including brain and spinal cord tissues, as a result of an autoimmune disease, preferably an autoimmune disease affecting the CNS. The invading leukocytes are primarily T-cells and monocytes, although other leukocytes may be present.

As used herein, “reducing leukocyte invasion” refers to decreasing the migration (i.e., invasion or infiltration) of leukocytes into tissues of the CNS, including brain and spinal cord tissues. Reducing leukocyte invasion also refers to reducing the cytotoxic affects mediated by leukocyte invasion, particularly with respect to the underlying neuronal cells and/or other supporting cells of the CNS tissue. Leukocyte invasion includes invasion by T-cells and monocytes. Reducing leukocyte invasion includes protecting CNS tissues from autoimmune attack. The cells of the CNS that are destroyed by leukocyte invasion include myelin-expressing cells and neighboring non-myelin expressing cells. Cell destruction may be by apoptosis, necrosis, or a combination, thereof. Reduced leukocyte invasion is characterized by such clinical indications as slowed disease progression, delayed onset or severity of morbidity, prolonged survival, improved quality of life, decreased or stabilized cognitive, motor, or behavioral symptoms. Reducing leukocyte invasion also includes preventing or reducing the risk of migration of leukocytes into tissues of the central nervous system (CNS). The reducing in leukocyte invasion may be partial or complete, for example, the reduction may be about 10%, about 20%, about 30%, about 40%, about 50%, about 60%, about 70%, about 80%, about 90%, or even about 95%, in comparative or actual reduction, as described herein.

As used herein, a “TrkB receptor agonist” or a “TrkB agonist” is a molecule that increases the amount of activation of TrkB receptors, producing effects similar to those produced by the naturally-occurring agonists BDNF and NT4. TrkB activation normally occurs by receptor-induced autophosphorylation, which initiates a characteristic cascade of intracellular signaling events. TrkB receptor agonists increase this activation, e.g., by modulating receptor dimerization or phosphorylation, by modulating the binding of naturally-occurring agonists, by mimicking the binding of naturally-occurring agonists, by causing the TrkB receptor to remain in an activated (e.g., phosphorylated) condition for a longer period of time (including indefinitely), or otherwise modulating TrkB activation or initiating the cascade of intracellular events that is characteristic of TrkB receptor activation. TrkB agonists include naturally-occurring agonist polypeptides, fragments, variants, and derivatives, thereof, including but not limited to the known TrkB agonists NT4 and BDNF. TrkB agonists include agonist antibodies, fragments, variants, and derivatives, thereof. Preferred properties of TrkB agonist are described herein. TrkB agonist of the invention may increase activation of TrkB by at least 5%, at least 10%, at least 20%, at least 30%, at least 50%, at least 100%, at least 200%, or more.

As used herein, “a fragment” polypeptide is a portion of a larger polypeptide that retains at least some of the biological properties or the larger polypeptide, such as the ability to activate TrkB receptors. Preferred fragments comprise the amino acid residues and/or structures or the larger polypeptide that resulted in the biological properties of the larger polypeptide. Polypeptide fragments may be called peptide, although no distinction is made herein between polypeptides and peptide. Exemplary fragments are described herein. Fragments may be derivativized as described herein.

As used herein, “a derivative” polypeptide has one or more covalent or non-covalent modifications, such as the addition or removal of a functional group or moiety. Examples of derivatives are provided herein.

As used herein, a “variant” of a particular polypeptide sequence is defined as a polypeptide sequence having at least 40% sequence identity to the particular polypeptide sequence over a certain length of one of the polypeptide sequences using an algorithm such as Clustal V or BLAST, e.g., the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

As used herein, “sequence identity,” as applied to polypeptide sequences, refer to the percentage of residue matches between at least two polypeptide sequences aligned using a standardized algorithm, such as Clustal V, MEGALIGN, or BLAST. Methods of polypeptide sequence alignment are well-known. Some alignment methods take into account conservative amino acid substitutions. Such conservative substitutions as described herein, and generally preserve the charge and hydrophobicity at the site of substitution, thus preserving the structure (and therefore function) of the polypeptide.

As used herein, “an amount effective for activating the TrkB receptor,” or similar expressions with respect to a TrkB agonist, refers to a quantity sufficient to increase in TrkB receptor activation (as defined herein and known in the art) compared to a baseline level of activation prior to the administration of the TrkB receptor agonist. The increase in activation may be at least 5%, at least 10%, at least 20%, at least 30%, at least 50%, at least 100%, at least 200%, or more. This amount will take into account such considerations as the route of administration, the half-life of the TrkB agonist in the body, the solubility, bioavailability, clearance rate, and other pharmacokinetic characteristics of the TrkB agonist, the body weight and metabolism of the animal or patient, etc. See also TrkB agonists.

As used herein, “an animal in need of treatment,” or similar expressions, means an animal, preferably a mammal including a human, having or at risk for developing an autoimmune disease involving the CNS. Examples of autoimmune diseases (or disorders, without distinction) affecting the CNS are experimental autoimmune encephalomyelitis (EAE) in mice, multiple sclerosis (MS) in humans, and similar autoimmune diseases found in other mammals. Autoimmune attack of the CNS is also observed in, e.g., immune rejection, optic neuropathies, inflammatory bowel disease, and Parkinson's disease.

As used herein, “naturally-occurring TrkB agonists” are molecules that exist in nature and function as activators of TrkB receptors. The known naturally-occurring agonists of TrkB receptors are the neurotrophins NT4 and BDNF. Naturally-occurring TrkB agonists include naturally-occurring variant molecules, such as a neurotrophins polypeptide expressed in an animal with a mutated TrkB allele.

An “isolated antibody”, as used herein, is intended to refer to an antibody that is substantially free of other antibodies having different antigenic specificities (e.g., an isolated antibody that specifically binds TrkB is substantially free of antibodies that specifically bind antigens other than TrkB). An isolated antibody that specifically binds TrkB may, however, have cross-reactivity to other antigens, such as TrkB molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

General Techniques

The present invention employs conventional techniques used in the fields of molecular biology, cell biology, biochemistry and immunology. Such techniques are described in references, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Cabs, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

TrkB Agonists

The invention provides methods of using TrkB agonists for the treatment of multiple sclerosis and other autoimmune disorders affecting the central nervous system (CNS). According to one feature of the invention, TrkB agonists are effective in reducing leukocyte invasion into tissues of the CNS and reducing the destruction of the underlying neuronal tissues of the CNS, and thus ameliorating the clinical manifestations of this disease, such as limb paralysis. In particular, TrkB agonists reduce the migration of monocytes and T-cells, which are active in presenting myelin antigens and administering cytotoxic effects on the cells producing them.

The observations leading to the invention were made using a well accepted animal model for MS, the experimental autoimmune encephalomyelitis (EAE) mouse. EAE is an experimental disease state that shares many clinical and pathological features with MS in humans. Many FDA-approved MS therapies were first discovered and developed based on the EAE models in mice and rats (reviewed by Steinman and Zamvil, 2006). EAE can be induced in C57BL/6 mice following immunization with peptide 35-55 of myelin oligodendrocyte glycoprotein (MOG) (Aharoni, R., et al., 2005a). Immunization with MOG induces myelin-specific autoimmune reactions, which cause demyelination and morbidity similar to that of MS.

Animal Experiments Using TrkB Agonists

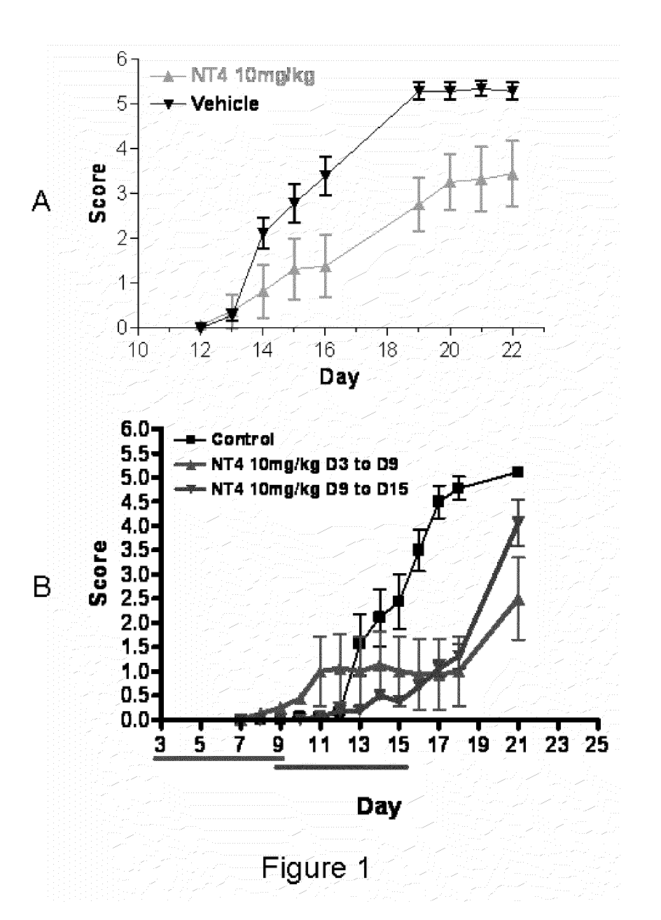

In experiments carried out in support of the present invention, direct administration of a TrkB agonist was shown to reduce morbidity and CNS tissue damage in animals with a CNS-specific autoimmune disease (FIGS. 1A and 1B). A recombinant form of the naturally occurring TrkB agonist NT4 was administered to EAE animals following MOG-induction (Example 5). The animals treated with NT4 showed significantly reduced morbidity compared to control animals. This result demonstrated that administration of a TrkB agonist was effective in slowing the progression of chronic EAE. Further experiments indicated that the protection afforded by NT4 when administered during an earlier time period is at least as good (if not better) than when administered during a later time period, with respect to the onset of symptoms, and that treatment with the TrkB agonist does not have to continue to the time of symptom onset in order for treatment to be effective (FIG. 1B). TrkB activation may be targeting steps relatively upstream in the induction of EAE.

Examples 1-4 and 6 describe TrkB antibodies, some of which functioned as TrkB agonists, based on their abilities to stimulate the biological activities of TrkB, i.e., activate TrkB (e.g., Example 6 and Table 1 and 2). Several antibodies, including antibody 38B8, were specific for TrkB and showed no significant binding to the other receptor tyrosine kinases assayed. Antibody 38B8 was even more selective for the TrkB receptor than the naturally-occurring agonists BDNF and NT4, which showed some cross-reactivity. A kinetic analysis of these ligand-receptor interactions is provided in Example 4 and Table 2.

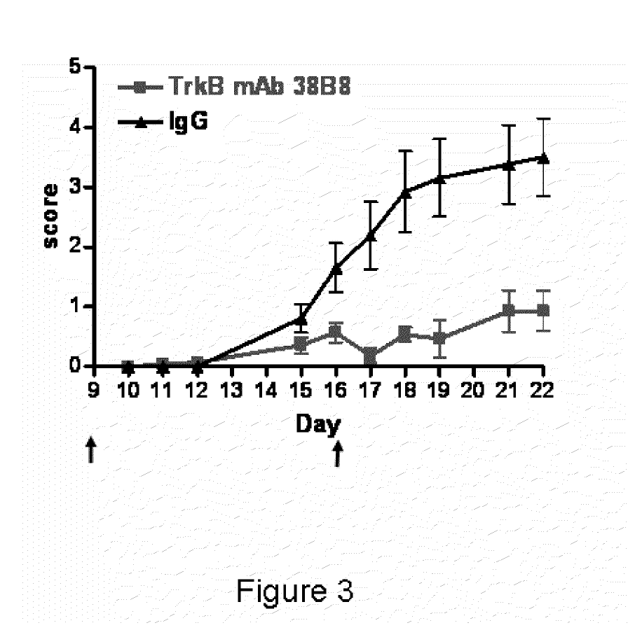

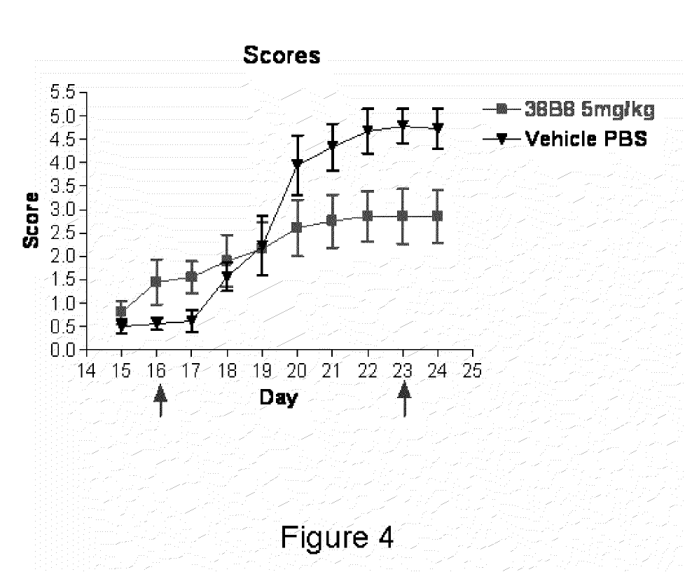

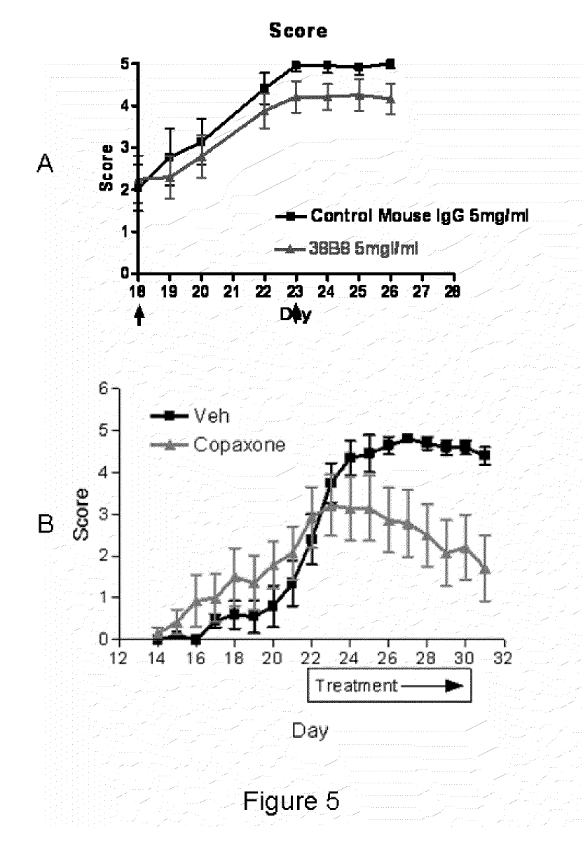

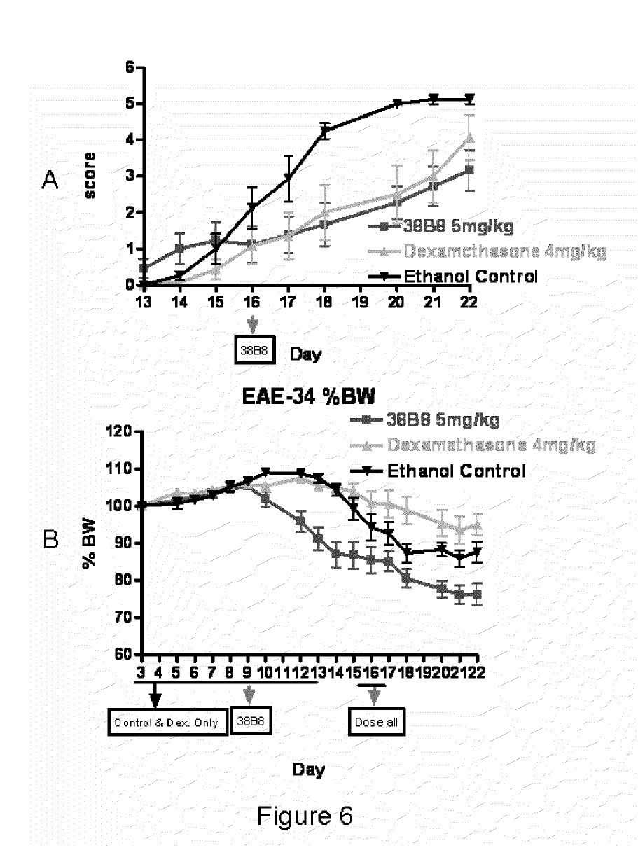

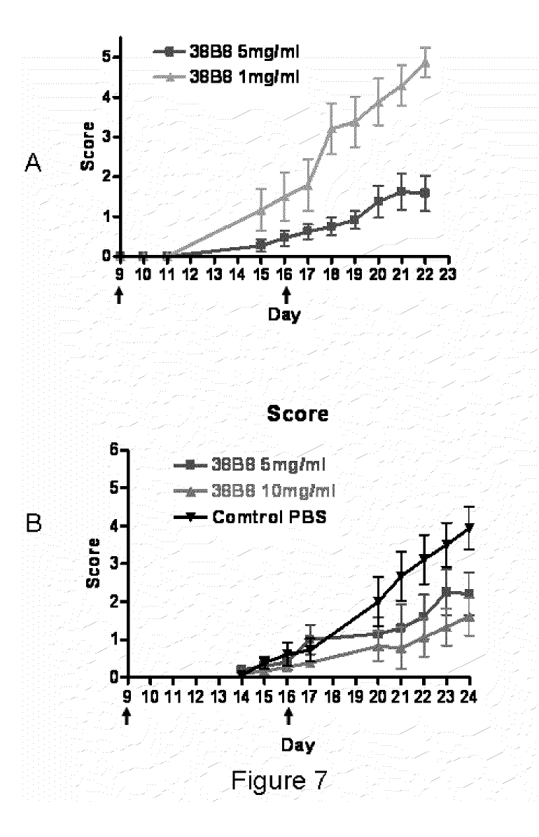

Animal experiments showed that a TrkB agonist antibody provided significant protection against EAE morbidity compared to a control immunoglobulin (FIG. 3, Example 7). The TrkB agonist antibody was effective when administered as late as 16 days following MOG-induction, and after the onset of clinical symptoms (FIG. 4). Comparison with glatiramer acetate (GA) suggested that the mechanism of action of the TrkB agonist is different from that of GA, and other current MS drugs (FIG. 5). Other results demonstrate that the beneficial effects of the TrkB agonist were similar to that of the immunosuppressant dexamethasone (FIGS. 6A and 6B). The beneficial effects of the TrkB agonist antibody were dose-dependent (FIGS. 7A and 7B).

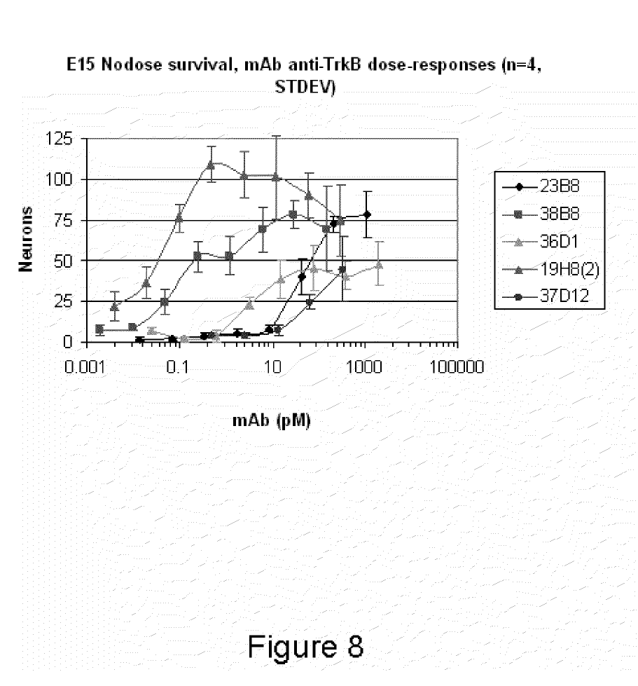

A neuron cell survival assay was used to demonstrate that TrkB agonist antibodies affect neuronal cells in a manner consistent with naturally-occurring TrkB agonists (Example 8). Adding increasing amounts of the TrkB agonist antibodies resulted in increased neuron survival (FIG. 8). The EC₅₀ values for TrkB agonist antibodies 38B8, 23B8, 36D1, and 37D12 in the neuron survival assays and in human KIRA assays are described in Example 1 and summarized in Table 1.

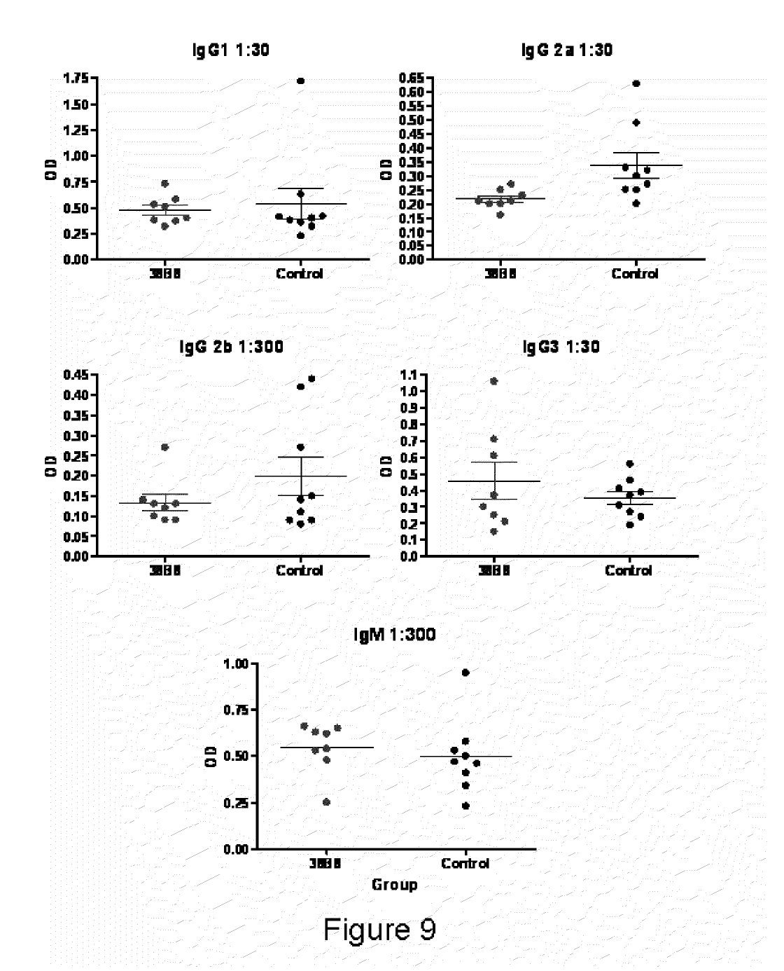

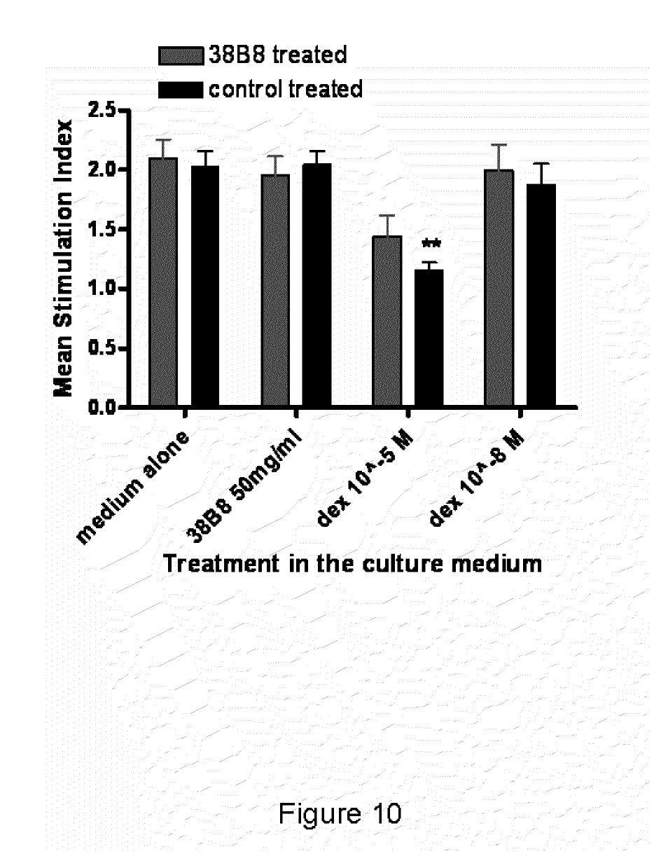

Current drugs for the treatment of MS (including GA and dexamethasone) are immunomodulators. A TrkB agonist antibody did not appear to function by suppressing the production of anti-MOG antibodies and, therefore, do not appear to function as conventional immunosuppressants (FIG. 9, Example 9). Furthermore, the presence of the TrkB agonist antibody had no apparent effect on splenocyte stimulation, as did the immunosuppressant dexamethasone (FIG. 10). Animals treated with the TrkB agonist retain the ability to produce normal anti-MOG T-cell and/or B-cell responses. These observations suggest that mechanisms of action of the immunosuppressant dexamethasone and TrkB agonists are different, and that the mechanism of action of TrkB agonists is not primarily at the level of leukocyte proliferation.

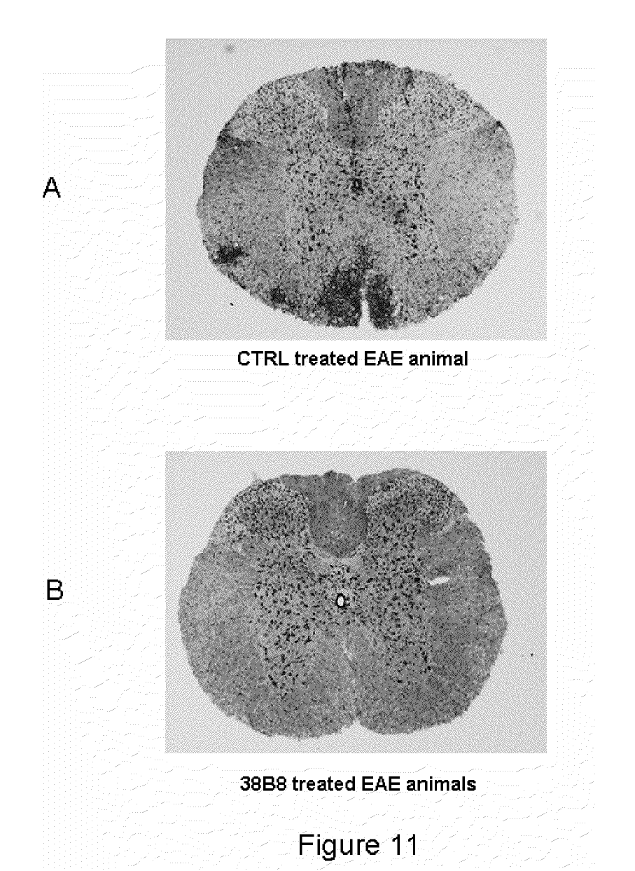

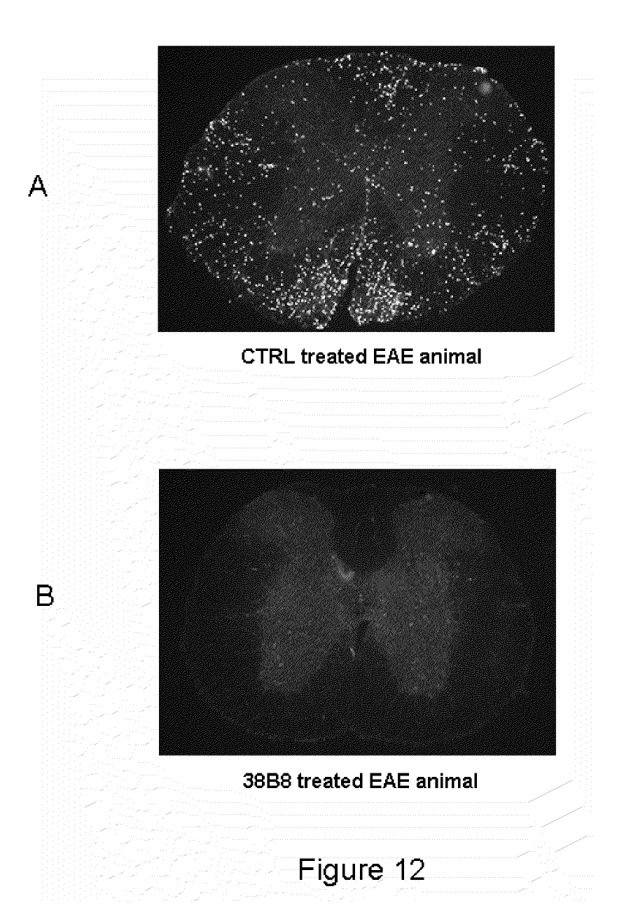

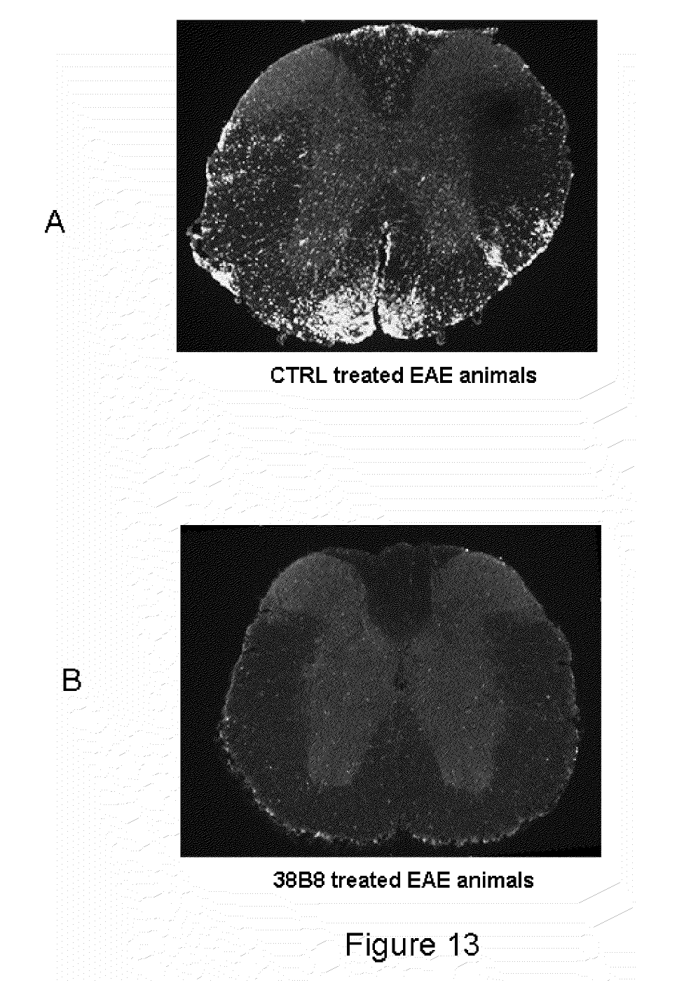

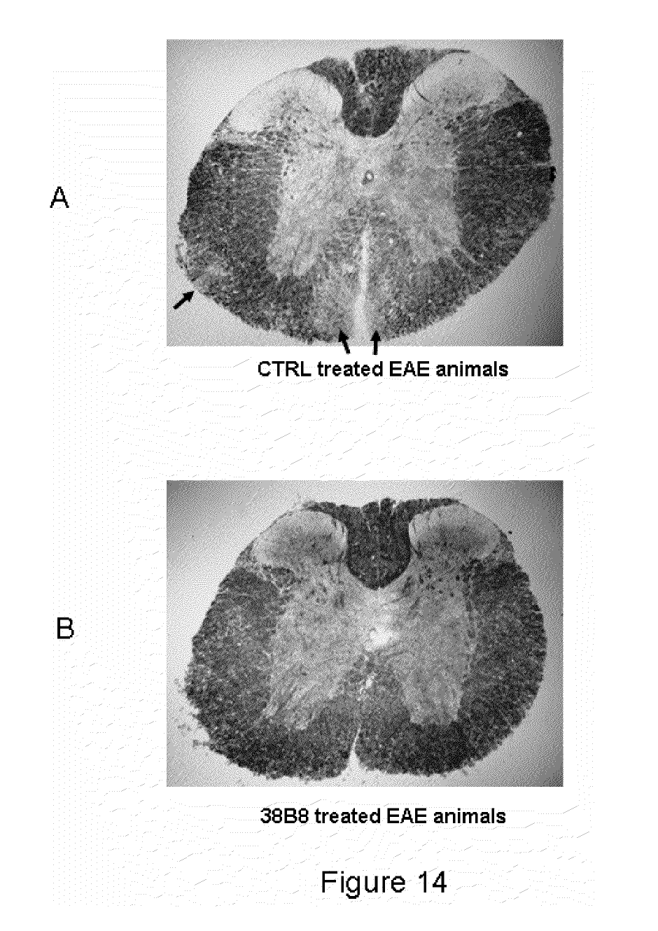

Histological analysis showed reduced leukocyte invasion in sections prepared from animals treated with a TrkB agonist. Some TrkB agonist-treated animals showed virtually no evidence of CNS leukocyte invasion (FIG. 11). Identification of the invading cells was performed by staining for CD3-expressing T-cells and CD68-expressing macrophages. Spinal cord tissues from TrkB agonist-treated animals show substantially reduced T-cell and monocyte invasion when compared to control animals (FIGS. 12 and 13). Regions of severe demyelination were apparent in control mice but not mice treated with the TrkB agonist (FIG. 14). The results of the histochemical staining of CNS tissue sections demonstrated that TrkB agonist treatment reduces lymphocyte and monocyte invasion of the CNS.

The results described above demonstrate that TrkB agonists are effective in reducing leukocyte invasion of the CNS and slowing the progression of EAE, a widely accepted animal model for MS. The naturally-occurring TrkB agonist NT4 and a TrkB agonist antibody were both effective in slowing disease progression. The agonist antibody demonstrated the greatest selectivity for TrkB and was used for further studies. The beneficial effects of TrkB agonists were dosage-dependent, with reduced morbidity being associated with increasing dosages of TrkB agonists. Unlike current MS drugs, TrkB agonists do not function primarily through immunosuppression. Histochemical experiments showed that TrkB agonists reduce invasion of CNS tissues by T-cells and monocytes.

TrkB Agonists for CNS Autoimmune Disorders

Without being limited to a theory, it is believed that TrkB agonists function primarily by modulating the migration of leukocytes. Cellular immunity may predominate in MS, making TrkB agonist a more effective type of drug than current immunosuppressants, which affect humoral immunity. Moreover, the present methods may be combined with current immunosuppressant treatments to produce additional therapeutic effects.

The TrkB agonists of the invention can be used to reduce leukocyte invasion of CNS tissues in a number of autoimmune or related diseases. In addition to being the model for MS, the EAE mouse is also used to study optic neuritis. TrkB agonists are expected to alleviate CNS immune invasion in all these diseases and other autoimmune disorders mediated by, inter alia, leukocyte invasion. Note that the terms disease and disorder are used without distinction.

A feature of the invention is the direct administration of a TrkB agonist to an animal suffering from an autoimmune disease. While the preferred embodiments of the invention are described in terms of polypeptides, the invention encompasses the administration of polynucleotides encoding such TrkB agonist polypeptides as will direct the expression of the encoded-TrkB agonists in the body. Methods of direct DNA injection and gene therapy delivery are known in the art. TrkB agonist polypeptides, or polynucleotides encoding them, are administered directly to an animal, as opposed to being induced by the administrations of a drug, such as GA. The invention also encompasses peptidomimetic molecules that bind and activate TrkB in a manner consistent with naturally-occurring TrkB agonists and/or agonist antibodies.

Particular TrkB agonists for use according to the methods described herein are described in further detail below. Additional TrkB agonists will be apparent to one skilled in the art without departing from the scope of the invention.

Naturally Occurring TrkB Agonists and their Derivatives

TrkB agonists include naturally-occurring agonist polypeptides, including but not limited to the known TrkB agonists NT4 and BDNF. NT4 and/or BDNF polypeptide sequences may be from the same species as the corresponding TrkB receptor or from a different species, provided that the resulting polypeptide binds to TrkB and functions as an agonist.

TrkB agonists include naturally-occurring and variant NT4 (i.e., NT-4/5 and similar names). NT4 polypeptides are described in U.S. Patent Application Publication Nos. 2005/0209148, 2003/0203383, and 2002/0045576, and in PCT WO 2005/08240. NT4 (i.e., NT4/5) polypeptides have been identified in a number of mammals. Amino acid substitutions of interest include G77 to K, H, Q or R; and R84 to E, F, P, Y or W. Protease cleavage sites may be removed to extend the half-life or NT4 and BDNF polypeptides, or added to allow the regulation of their activity. TrkB agonists may be conjugated or fused to half-life extending moieties, such as a PEG, the IgG Fc region, albumin, or a peptide or epitopes such as Myc, HA (hemagglutinin), His-6, or FLAG.

BDNF polypeptides have also been identified in a number of mammals (see, e.g., U.S. Pat. No. 5,180,820 and U.S. Patent Application Publication No. 2003/0203383).

Naturally-occurring and variant NT4 and BDNF polypeptides of the invention include chimeras, variants, fragments (including peptides), and/or derivatives thereof. Preferred fragments include the TrkB-binding portion of a naturally-occurring polypeptides, or a chimeric, consensus, or mutated equivalent binding portion. Fragments include synthetic peptides. Variants include naturally-occurring amino acid sequence variants having conservative and non-conservative amino acid substitutions.

Conservative substitutions involve amino acid residues of similar size, charge, or hydrophobicity. For example, Ala may be substituted by Val, Leu, or Ile. Arg may be substituted by Lys, Gln, or Asn. Asn may be substituted by Gln, His, Lys, or Arg. Asp may be substituted by Glu. Cys may be substituted by Ser. Gln may be substituted by Asn. Glu may be substituted by Asp. Gly may be substituted by Pro. His may be substituted by Asn, Gln, Lys, or Arg. Ile may be substituted by Leu, Val, Met, Ala, Phe, or Norleucine. Leu may be substituted by Norleucine, Ile, Val, Met, Ala, or Phe. Lys may be substituted by Arg, Gln, or Asn. Met may be substituted by Leu, Phe, or Ile. Phe may be substituted by Leu, Val, Ile, or Ala. Pro may be substituted by Gly. Ser may be substituted by Thr. Thr may be substituted by Ser. Trp may be substituted by Tyr. Tyr may be substituted by Trp, Phe, Thr, or Ser. Val may be substituted by Ile, Leu, Met, Phe, Ala, or Norleucine.

Substantial modifications in function may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side chain properties (some of these may fall into several functional groups):
(1) Hydrophobic: Met, Ala, Val, Leu, Ile, Norleucine;
(2) Neutral hydrophilic: Cys, Ser, Thr;
(3) Acidic: Asp, Glu;
(4) Basic: Asn, Gln, His, Lys, Arg;
(5) Aromatic: Trp, Tyr, Phe; and
(6) Residue that induce bending: Gly and Pro.

Non-conservative substitutions exchange a member of one class for a member of another class, or involve a substitution not identified as conservative in the previous paragraphs.

Variants include naturally-occurring amino acid sequence variants and engineered variants, provided that the resulting polypeptides or derivatives bind to TrkB and function as agonists. Assays for measuring TrkB activation are described herein and in the references cited.

Further variants include TrkB agonist polypeptides having partial amino acid sequences from other related ligands of the Trk family of tyrosine receptor kinases, including NT3 and NGF. Variants further include polypeptides having consensus Trk or consensus receptor tyrosine kinase binding and/or activation sequences.

Other derivatives include covalently and non-covalently modified peptides and polypeptides (e.g., acylated, pegylated, farnesylated, glycosylated, or phosphorylated) polypeptides. Particular pegylated and other modified forms of NT4 are described in U.S. Patent Application Publication No. 2005/0209148. The polypeptides may include additional functional groups to modulate binding and/or activity, allow imaging in the body, modulate half-life, modulate transport across the blood-brain barrier, or assist in the targeting of the polypeptide to a particular cell type or tissue. The polypeptides may comprise amino acid substitutions to facilitate modification (e.g. the addition of pegylation, glycosylation, or other sites), provided that the substitutions do not substantially affect the binding of the polypeptide to TrkB or agonist activity.

A common modification is pegylation to reduce systemic clearance with minimal loss of biological activity. Polyethylene glycol polymers (PEG) may be linked to various functional groups of the NT4 and BDNF polypeptides (as well as TrkB agonist antibodies) using methods known in the art (see, e.g., Roberts et al. (2002), Advanced Drug Delivery Reviews 54:459-476; Sakane et al. (1997) Pharm. Res. 14:1085-91).

PEG may be linked to, e.g., amino groups, carboxyl groups, modified or natural N-termini, amine groups, and thiol groups. In some embodiments, one or more surface amino acid residues are modified with PEG molecules. PEG molecules may be of various sizes (e.g., ranging from about 2 to 40 kDa). PEG molecules linked to NT4, BDNF, or other polypeptides may have a molecular weight about any of 2000, 10,000, 15,000, 20,000, 25,000, 30,000, 35,000, 40,000 Da. PEG molecule may be a single or branched chain. To link PEG to TrkB agonist polypeptides, a derivative of the PEG having a functional group at one or both termini may be used. The functional group is chosen based on the type of available reactive group on the polypeptide. Methods of linking derivatives to polypeptides are known in the art.

Pegylated NT4 has been generated and shown to function as NT4 in animals (see e.g., Examples 6 and 7 of U.S. Patent Application Publication No. 2005/0209148 and PCT WO 2005/082401). The serine residue at position 50 of the mature human NT4 may be changed to cysteine to generate NT4-S50C which is then pegylated, wherein the PEG is linked to the cysteine at position 50. One example of an N-terminal specific attachment for PEG is to mutate the residue at position 1 to a serine or threonine to facilitate pegylation. Similar methods apply to BDNF and other polypeptides for use in the invention.

Polypeptides or their derivatives may be linked to other molecules directly or via synthetic linkers. Preferred TrkB agonist polypeptides, fragments, or derivatives, thereof, exhibit similar or better binding affinity, selectivity, and activation compared to naturally-occurring TrkB agonists for which values have been reported. Small portions of the agonist polypeptides may be referred to as “peptides,” although this terminology should not be construed as limiting.

Preferred TrkB agonists exhibit similar biological properties compared to BDNF and NT4 in the numerous experiments and assays described herein, including the kinetic assays, the EAE animal model, the KIRA assay, and the neuron survival assay.

TrkB Antibody Agonists and their Derivatives

TrkB agonists include agonist antibodies, fragments, variants, and derivatives, thereof. Suitable agonist antibodies are selective for TrkB and bind with affinities similar to or greater than naturally-occurring NT4 and BDNF polypeptides. However, the long circulating half-lives of antibodies compared to naturally-occurring TrkB agonists, makes the binding affinity less critical. The binding affinity of antibody 38B8 was determined to be 46±10 nM (see above and Example 4). Preferred TrkB agonist antibodies have a Kd of less than 100 nM, less than 10 nM, less than 1 nM, less than 100 pM, or even less than 10 pM, using the particular assay conditions described in Example 4.

Preferred TrkB agonists also exhibit similar biological properties compared to antibody 38B8 (and naturally-occurring agonists) in the numerous experiments and assays described herein, including the binding assays, the EAE animal model, the KIRA assay, and the neuron survival assay. For example, preferred TrkB agonists exhibit an EC₅₀ value in the neuron survival assay described in Example 1 (including Table 1) of less than 11 pM. Preferred TrkB agonists have an EC₅₀ value of from about 1 pM to about 10 pM, from about 0.1 pM to about 1 pM, from about 0.01 pM to about 0.1 pM, or even lower than 0.01 pM. Exemplary EC₅₀ values are 0.2 pM for 38B8 and 5 pM for 36D1. Preferred TrkB agonists also exhibit similar biological properties compared to antibody 38B8 using the KIRA assay (Example 1, including Table 1). Preferred TrkB agonists have a EC₅₀ value in the KIRA assay of less than 50 nM, preferably from about 5 nM to less than 50 nM, from about 0.5 nM to about 5 nM, or even lower than 0.5 nM. An exemplary EC₅₀ value is about 5 nM, under the assay conditions provided.

TrkB agonist antibodies include polyclonal antibodies, monoclonal antibodies, recombinant antibodies, hybrid, consensus, chimeric, bispecific, or conjugated antibodies. The antibodies may be of any isotype (i.e., IgA, IgD, IgE, IgG, or IgM), if applicable. Antibodies include antibody fragments (e.g., Fab, Fab′, F(ab′)2, Fv, Fc, and single chain (ScFv) antibodies). A suitable TrkB agonist antibody, or a functional fragment or derivative thereof, binds to and activates TrkB in the manner described herein, for example, with respect to the monoclonal antibody agonist 38B8. TrkB agonist antibodies include antibody fragments, which encompass polypeptides comprising amino acid sequences that were derived from antibodies. Of particular importance are amino acid sequences involved in antigen recognition, such as those in the CDR region.

Monoclonal antibody and mouse hybridoma methods are well-known in the art. The use of nonhuman antibodies for the treatment of humans may be tolerated in combination with immunosuppressive drugs. Such drugs are routinely administered to treat or reduce the symptoms of MS and other autoimmune and inflammatory diseases. Immunoregulatory drugs are known in the art and include glucocorticoids, cytostatic agents (e.g., alkylating agents, antimetabolites, methotrexate, azathioprine and mercaptopurine), cytotoxic antibodies (e.g., T-cell receptor and IL-2-specific antibodies), drugs that act on immunophilins (e.g., cyclosporine, tacrolimus, sirolimus, rapamicin, RAPAMUNE, PROGRAF, and FK506), interferons (e.g., IFN-β), opioids, TNF-binding proteins (e.g., circulating receptors), mycophenolate, and other biological agents used to suppress an animal's immune responses to foreign antibodies or therapeutic antigens.

Methods for humanizing monoclonal antibodies derived from a different species, e.g., mice, are well known in the art. For the treatment of humans, human or humanized antibodies are preferred. Humanized antibodies comprise a minimal number of non-human amino acid sequences such that they are minimally immunogenic, or non-immunogenic in humans. Preferred humanized antibodies are not recognized as foreign by the human immune system. Such antibodies may be chimeric immunoglobulins, immunoglobulin fragments (e.g., Fv, Fab, Fab′, F(ab′)₂ or other portions of immunoglobulin chains comprising the amino acid residues necessary for antigen-binding, with the majority of the amino acid sequences outside the antigen binding site being derived from human immunoglobulins. Amino acid residues within the complementarity determining region (CDR) may also be substituted with human-specific residues, so long as antigen binding is not adversely affected.

A human antibody is an antibody exclusively or substantially comprising amino acid sequences of human antibodies. Human antibodies also includes antibodies comprising at least one human heavy chain polypeptide or at least one human light chain polypeptide, or substantial fragment, thereof, optionally in combination with heavy or light chains from another animal. One such example is an antibody comprising murine light chain and human heavy chain polypeptides.

Suitable TrkB agonist antibodies, fragments, or derivatives, thereof may be expressed or secreted in transgenic animals, mammalian cells (including B-cells), avian cells, insect cells, yeast, or bacteria. For example, suitable human antibodies may be generated by immunizing transgenic animals (e.g. mice) capable of producing fully human or substantially human immunoglobulins. Antibodies can also be produced by means of gene-shuffling, phage display, E. coli display, ribosome display, mRNA display, protein fragment complementation, or RNA-peptide screening. Methods for designing and producing humanized and human antibodies and their derivatives are described in, e.g., U.S. Pat. Nos. 7,005,504, 6,800,738, 6,407,213, 6,054,297, 6,331,415, and 5,750,373 (Genentech); U.S. Pat. Nos. 6,833,268, 6,207,418, 6,114,598, and 6,075,181 (Abgenix); U.S. Pat. No. 6,498,285 (Alexion); U.S. Pat. Nos. 7,074,557 5,885,793, 5,837,242, 5,733,743, 5,565,332 (Cambridge Antibody Technology); U.S. Pat. Nos. 7,118,879, 6,979,538, 6,326,155, 5,994,125, 5,837,500 (Dyax); U.S. Pat. Nos. 6,753,136, 6,667,150, 6,300,064, 5,514,548 (MorphoGen/Protein Design Labs); and 6,461,824, 6,204,023, 5,821,123, 5,595,898, 5,576,184, 4,698,420 (Xoma); U.S. Pat. Nos. 7,041,870, 6,680,209, 6,500,931, 6,111,166, 6,096,311, 6,071,517, 6,063,116 (Medarex); and U.S. Pat. No. 4,816,397 (Celltech). Methods for producing human or humanized antibodies are also described in Vaughan et al. (1996) Nature Biotechnology 14:309-14; Sheets et al. (1998) Proc. Nat'l. Acad. Sci. USA 95:6157-6162; Hoogenboom and Winter (1991) J. Mol. Biol., 227:381; Marks et al. (1991) J. Mol. Biol., 222:581; Cole et al. (1985) in Monoclonal Antibodies and Cancer Therapy (Alan R. Liss) p. 77; and Boerner et al., 1991, J. Immunol., 147 (1):86-95.

TrkB agonist antibodies may be covalently and non-covalently modified (e.g., acylated, pegylated (see, e.g., Example 5), farnesylated, glycosylated, phosphorylated, etc.) and may include additional functional groups to modulate binding, modulate agonist activity, allow imaging of the polypeptide in the body, modulate the half-life of the polypeptide, or modulate transport across the blood-brain barrier. The polypeptides may comprise amino acid substitutions to facilitate modification (e.g. the additional pegylation, glycosylation, or other sites), provided that the substitutions do not substantially affect the binding of the polypeptide(s) to TrkB or agonist activity. Polypeptides or their derivatives may be linked to other molecules directly or via synthetic linkers.

Suitable antibody fragments or derivatives comprise amino acid residues that mediate TrkB-binding and activation, as described herein and in the references cited. Antibody specificity is primarily determined by residues in six small loop regions, known as complementarity determining regions (CDRs) or hypervariable loops, located near the N-terminus of light and heavy chains. The CDRs in light chains are generally between amino acid residues 24 and 34 (CDR1-L), 50-56 (CDR2-L), and 89-97 (CDR3-L). The CDRs in heavy chains are generally between amino acid residues 31 and 35b (CDR1-H), 50-65 (CDR2-H), and 95-102 (CDR3-H). The length of some CDRs is more variable than others. CDR1-L varies in length from about 10-17 residues, while CDR3-H varies in length from about 4-26 residues. Other CDRs are of fairly standard lengths. Padlan, E. A., et al. (1995) FASEB. J. 133-39. Preferred TrkB agonist antibody fragments for use in the invention comprise amino acid residues from the CDR or from heavy and/or light chain domains within the CDR.

Antibodies for use with the invention include naturally-occurring amino acid sequence variants having conservative and non-conservative amino acid substitutions, as described above and known in the art.

Preferred TrkB agonist antibodies, fragments, or derivatives, thereof, exhibit similar or better binding affinity, selectivity, and activation ability, compared to naturally-occurring TrkB agonists. Small portions of the agonist polypeptides may be referred to as “peptides,” although this terminology should not be construed as limiting.

TrkB Agonist Formulations

TrkB agonists can be used in the manufacture of a medicament for the treatment of an autoimmune disease affecting the central nervous system, such as multiple sclerosis. In this manner, compositions comprising TrkB agonists may be used to treat a disease in a mammal (including a human patient), as defined herein. TrkB agonist compositions may further comprise suitable pharmaceutically acceptable excipients, which are known in the art. Generally, TrkB agonist compositions are formulated for administration by injection (e.g. intraperitoneal, intravenous, subcutaneous, intramuscular, etc.), although other forms of administration can be used. TrkB agonists can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Suitable carriers, diluents and excipients are well known in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament). Some formulations may include carriers such as liposomes. Liposomal preparations include, but are not limited to, cytofectins, multilamellar vesicles and unilamellar vesicles. Excipients and formulations for parenteral and nonparenteral drug delivery are set forth in Remington, The Science and Practice of Pharmacy (2000).

Administration and Dosing

The administration schedules and therapeutic dosages exemplified herein were based on such factors as animal body mass, the half-life of TrkB agonist in the blood, and the affinity of the TrkB agonists. Preferred administration schedules and dosages maintain a therapeutic amount of TrkB agonists in the body between administrations. Effective dosage ranges for NTs (including naturally-occurring TrkB agonists) are exemplified herein, in Davies et al. (1993), and in other references. Effective dosage ranges for agonist antibodies are exemplified herein (e.g., 1-10 mg/kg in EAE animals), and will provide a starting point for determining the optimum dosage for similar agonist antibodies.

As demonstrated by experiments performed in support of the invention (see, e.g., FIGS. 1B, 3, and 4), a “pulse treatment” early in the course of the disease appears to be sufficient to reduce morbidity in animals. Continuing administration of the TrkB agonist may not be required for the efficacy of the treatment.

The particular dosage regimen, i.e., dose, timing and repetition, will depend on the age, condition, and body weight of the human or animal to be treated. Initial dosage regimens may be extrapolated from animal experiments. The timing/frequency of TrkB agonist administrations should be based on the circulating half-life (or the half-life in neuronal tissue) of a particular TrkB agonist, the amount of agonist that passes the blood-brain barrier, the half-life of the agonist in cells, toxicity, and side-effects, as they apply.

In the present studies, administration of TrkB agonists was performed by intraperitoneal (i.p.) injection; however, other routes of administration are expected to be effective (e.g., intravenous, subcutaneous, intramuscular). Intracranial or intraspinal administration (or administration to other tissues of the CNS) is likely to be effective. Other routes of administration may be suitable, depending on the particular TrkB agonist, its coating, conjugation, or particular biological properties (e.g., oral, sublingual, intrasynovial, mucosal, transdermal, intra-articular, vaginal, anal, intraurethral, nasal, aural, via inhalation, insufflation, via catheter, as a bolus, on a stent or other implantable device, in an embolic composition, in an intravenous drip, on a patch or dissolving film, etc.).

The TrkB agonist is preferably administered via a suitable peripheral route. Nonetheless, it is understood that a small percentage of the agonist may traverse the blood-brain barrier and be delivered to cells of the central nervous system. In some cases, the amount of peripherally administered TrkB agonist that gains access to the CNS is small (even less than 1%).

A feature of the invention is the direct administration of a TrkB agonist to a mammalian subject suffering from an autoimmune disease. “Direct” means that TrkB agonist polypeptides, or polynucleotides capable of directing the expression of such polypeptides, are delivered to an animal by a standard route of inoculation. TrkB agonists may be delivered to animals in combination with other pharmacological agents, including immunosuppressants such as glucocorticoids, cytostatic agents (e.g., alkylating agents, antimetabolites, methotrexate, azathioprine and mercaptopurine), cytotoxic antibodies (e.g., T-cell receptor and IL-2-specific antibodies), drugs that act on immunophilins (e.g., cyclosporine, tacrolimus, sirolimus, rapamicin), interferons (e.g., IFN-β), opioids, TNF-binding proteins (e.g., circulating receptors), mycophenolate, and other biological agents used to suppress an animal's immune responses to foreign antibodies or therapeutic antigens.

An advantage of the TrkB agonist antibody over naturally-occurring TrkB agonists is that antibodies tend to have relatively long circulating half-lives compared to circulating protein-ligands. For example, while naturally-occurring agonists may require daily administration, antibodies may only require weekly administration. Another advantage is that antibodies tend to have higher binding affinities and are more selective for their antigens than are cell receptors for their protein ligands.

Treatment with TrkB agonists can be combined with conventional treatments for multiple sclerosis and related disorders. Conventional drugs for the treatment and management of multiple sclerosis include but are not limited to: ABC (i.e., Avonex-Betaseron/Betaferon-Copaxone) treatments (e.g., interferon beta 1a (AVONEX, REBIF), interferon beta 1b (BETASERON, BETAFERON), and glatiramer acetate (COPAXONE); chemotherapeutic agents (e.g., mitoxantrone (NOVANTRONE), azathioprine (IMURAN), cyclophosphamide (CYTOXAN, NEOSAR), cyclosporine (SANDIMMUNE), methotrexate, and cladribine (LEUSTATIN); corticosteroids and adreno-corticotrophic hormone (ACTH) (e.g., methylprednisolone (DEPO-MEDROL, SOLU-MEDROL), prednisone (DELTASONE), prednisolone (DELTA-CORTEF), dexamethasone (MEDROL, DECADRON), adreno-corticotrophic hormone (ACTH), and corticotrophin (ACTHAR); pain mediation (dysaesthesia) (e.g., carbamazepine (TEGRETOL, EPITOL, ATRETOL, CARBATROL), gabapentin (NEURONTIN), topiramate (TOPAMAX), zonisamide (ZONEGRAN), phenyloin (DILANTIN), desipramine (NORPRAMIN), amitriptyline (ELAVIL), imipramine (TOFRANIL, IMAVATE, JANIMINE), doxepin (SINEQUAN, ADAPIN, TRIADAPIN, ZONALON), protriptyline (VIVACTIL), cannabis and synthetic cannabinoids (MARINOL), pentoxifylline (TRENTAL), ibuprofen (NEUROFEN), aspirin, acetaminophen, and hydroxyzine (ATARAX); and other treatments (e.g., natalizumab (ANTEGREN), alemtuzumab (CAMPATH-1H), 4-aminopyridine (FAMPRIDINE), 3,4 diaminopyridine, eliprodil, IV immunoglobin (GAMMAGARD, GAMMAR-IV, GAMIMUNE N, IVEEGAM, PANGLOBULIN, SANDOGLOBULIN, VENOGLOBULIN), pregabalin, and ziconotide).

Kits of Parts

The invention also provides kits of parts (kits) for practicing the methods of the invention. Kits include a suitably isolated and sterilized TrkB agonist and instructions for use in accordance with any of the methods of the invention described herein. Generally, these instructions comprise a description of how to administer the TrkB agonist. The kit may further comprise instructions for identifying animals in need of treatment and for monitoring or measuring the effectiveness of treatment. The instructions generally include information relating to dosage, dosage scheduling (frequency of administration), and route of administration. The instructions supplied in the kits may be written or machine/computer readable as in the form of a data file or spreadsheet.

The kits may also comprise an apparatus for administering TrkB agonists, including syringes, needles, catheters, inhalers, pumps, alcohol swaps, gauze, CNS biopsy apparatus, histological antibodies and stains, etc. The components of the kit are sterilized as required. Kits may also provide additional pharmaceutical agents, including but not limited to immunosuppressants, such as GA and dexamethasone. Kits may include date stamps, tamper-proof packaging, and radio frequency identification (RFID) tags or other inventory control features.

### EXAMPLES

The following examples are provided to further illustrate the invention. Additional aspects of the invention will be apparent to one skilled in the art without departing from the scope of the invention.

### Example 1

### Generation and Screening of TrkB Agonist Antibodies

Immunization for Generating Monoclonal Anti-TrkB Agonist Antibodies

A Balb/C mouse was injected 5 times on a regular schedule with 8 μg of human TrkB extracellular domain as antigen. His-tagged human TrkB extracellular domain (residues 31-430) was expressed using vector pTriEx-2 Hygro (Novagen, Madison Wis.) in 293 cells. TrkB extracellular domain was purified using Ni-NTA resin via manufacturers instructions (Qiagen, Valencia, Calif.). For the first 4 injections, antigen was prepared by mixing human TrkB with RIBI adjuvant system and alum. A total of 8 μg of antigen was given via injection to the scruff of the neck, the foot pads and IP, approximately every 3 days over the course of 11 days. On Day 13, the mouse was euthanized and the spleen was removed. Lymphocytes were fused with 8653 cells to make hybridoma clones. Clones were allowed to grow then selected as anti-TrkB positives by ELISA screening with both human and rat TrkB ELISA.

ELISA Screening Anti-TrkB Antibodies:

Supernatants from growing hybridoma clones were screened for their ability to bind both human and rat TrkB. The assays were performed with 96-well plates coated overnight with 100 μl of 0.5 μg/ml rat or human TrkB-Fc fusion protein. Excess reagents were washed from the wells between each step with PBS containing 0.05% Tween-20. Plates were then blocked with phosphate buffered saline (PBS) containing 0.5% BSA. Supernatant was added to the plates and incubated at room temperature for 2 hours. Horse radish peroxidase (HRP) conjugated goat-anti mouse Fc was added to bind to the mouse antibodies bound to TrkB. Tetramethyl benzidine was then added as substrate for HRP to detect amount of mouse antibody present in the supernatant. The reaction was stopped and the relative amount of antibody was quantified by reading the absorbance at 450 nm. Fifty antibodies were shown positive in the ELISA assay. Among these antibodies, five were further tested and shown to have agonist activity. See Table 2 below.

KIRA Assay:

This assay was used to screen antibodies found positive in the ELISA for the ability to induce receptor tyrosine kinase activation for human TrkB. Sadick, et. al. (1997) Experimental Cell Research 234:354-61. Utilizing a stable cell line transfected with gD tagged human TrkB, purified murine antibodies from the hybridoma clones were tested for their ability to activate the receptor on the surface of the cells similar to the activation seen with the natural ligands, BDNF and NT-4/5. Natural ligand induced self phosphorylation of the kinase domain of the TrkB receptor. After the cells were exposed to various concentrations of the antibodies, they were lysed and an ELISA was performed to detect phosphorylation of the TrkB receptor. EC50 (shown in Table 2 below and FIG. 10) was determined for each putative TrkB agonist and was compared to that of the natural ligand NT-4/5.

E15 Nodose Neuron Survival Assay:

The Nodose ganglion neurons obtained from E15 embryos were supported by BDNF, so that at saturating concentrations of the neurotrophic factor the survival was close to 100% by 48 hours in culture. In the absence of BDNF, less than 5% of the neurons survived by 48 hours. Therefore, the survival of E15 nodose neurons is a sensitive assay to evaluate the agonist activity of anti-TrkB antibodies, i.e. agonist antibodies will promote survival of E15 nodose neurons.

Time-mated pregnant Swiss Webster female mice were euthanized by CO₂ inhalation. The uterine horns were removed and the embryos at embryonic stage E15 were extracted. The nodose ganglia were dissected then trypsinized, mechanically dissociated and plated at a density of 200-300 cells per well in defined, serum-free medium in 96-well plates coated with poly-L-ornithine and laminin. The agonist activity of anti-TrkB antibodies was evaluated in a dose-response manner in triplicates with reference to human BDNF. After 48 hours in culture the cells were subjected to an automated immunocytochemistry protocol performed on a Biomek FX liquid handling workstation (Beckman Coulter). The protocol included fixation (4% formaldehyde, 5% sucrose, PBS), permeabilization (0.3% Triton X-100 in PBS), blocking of unspecific binding sites (5% normal goat serum, 0.1% BSA, PBS) and sequential incubation with a primary and secondary antibodies to detect neurons. A rabbit polyclonal antibody against the protein gene product 9.5 (PGP9.5, Chemicon), which was an established neuronal phenotypic marker, was used as primary antibody. Alexa Fluor 488 goat anti-rabbit (Molecular Probes) was used as secondary reagent together with the nuclear dye Hoechst 33342 (Molecular Probes) to label the nuclei of all the cells present in the culture. Image acquisition and image analysis were performed on a Discovery-1/GenII Imager (Universal Imaging Corporation). Images were automatically acquired at two wavelengths for Alexa Fluor 488 and Hoechst 33342, with the nuclear staining being used as reference point, since it is present in all the wells, for the image-based auto focus-system of the Imager. Appropriate objectives and number of sites imaged per well were selected to cover the entire surface of each well. Automated image analysis was set up to count the number of neurons present in each well after 48 hours in culture based on their specific staining with the anti-PGP9.5 antibody. Careful thresholding of the image and application of morphology and fluorescence intensity based selectivity filters resulted in an accurate count of neurons per well. EC50s (shown in Table 1 below and FIG. 8) were determined for each putative TrkB agonist antibody and were compared to that of the natural ligands.

The following Table shows the five anti-TrkB antibodies identified and their activities on mouse neuron survival and phosphorylation activity on human TrkB.

TABLE 1
Effects of five anti-TrkB antibodies on neuron survival
and phosphorylation

| | | | | Human | |
|---|---|---|---|---|---|
| | | | Mouse Neuron | KIRA | Effect on |
| | HuTrkB | RatTrkB | survival Assay | Assay | mouse |
| Clone | ELISA | ELISA | (estimated EC₅₀) | (EC₅₀) | weight |
| 18H6 | + | + | 0.01 pM | 0.5 nM | Not |
| | | | | | tested |
| 38B8 | + | + | 0.2 pM | 5 nM | Reduce |
| 36D1 | + | + | 5 pM | 5 nM | Reduce |
| 37D12 | + | + | 50 pM | 56 nM | No |
| | | | | | change |
| 23B8 | + | + | 11 pM | 50 nM | No |
| | | | | | change |

Intracranial Injections of Anti-TrkB Agonist Antibodies to Mice:

Male C57B6 retired breeder mice (aged 8-12 months) were obtained from Charles River Laboratories (Hollister facility) and allowed to acclimate in a temperature/humidity-controlled environment, with a 12 hour light/dark cycle, with ad libitum access to food and water for at least 5 days before injection. Each mouse was anaesthetized with isoflurane, to clip a section of hair above the skull. The mouse was fixed onto the stereotaxic surgery instrument (Kopf model 900), anaesthetized, and kept warm with an electric heating pad set to medium. Betadine was rubbed onto the shaved portion of the skull to sterilize the region. A small median-longitudinal incision of about 1 cm long was made above the cranium starting just behind the ears towards the eyes. The skull was revealed, and a circular space of about 1 cm in diameter of the skull surface was cleaned with a cotton swab to remove any connective tissue. The surface was cleaned with a cotton swab dipped in 30% hydrogen peroxide, to reveal the Bregma. Using the drill tip as a probe to measure skull depth, the cranium was adjusted horizontally and vertically to insure that it was level before drilling. Deviation of depth (zeroed at the Bregma), from 0.5 mm medial compared to 0.5 mm lateral, as well as 0.5 mm anterior compared to 0.5 mm posterior, was minimized to within a difference of ±0.05 mm. According to the mouse brain atlas (Franklin, K. B. J. & Paxinos, G., The Mouse Brain in Stereotaxic Coordinates. Academic Press, San Diego, 1997), coordinates for a single, lateral, intrahypothalamic injection were as follows: 1.30 mm posterior from the Bregma; −0.5 mm from midline; Depth, 5.70 mm from the surface of the skull (at the Bregma). A small hole was drilled through the skull, avoiding contact with the brain. The drill was replaced with a beveled 26 gauge needle attached to a Hamilton syringe (model 84851) and returned to the same coordinates. 2 μl of compound was injected into the lateral hypothalamus incrementally over the course of 2 minutes. The needle was kept at this position for 30 seconds after injection, then raised 1 mm. After another 30 seconds, the needle was raised an additional 1 mm. 30 seconds later, the needle was completely removed. The incision was then closed and held together with 2-9 mm wound clips (Autoclip, Braintree Scientific, Inc.). The injection was performed on day 0. Body weight and food intake were monitored daily until day 15.

As shown in Table 1 (above), intracranial injections of antibody 38B8 and 36D1 at the specified dose significantly reduced body weight and food intake in mice. The control IgG antibody and 23B8, given at the specified dose, did not significantly affect either the food intake or the body weight. The murine hybridoma strain that produces antibody 38B8 was deposited at the American Type Culture Collection, 10801 University Boulevard, Manassas, Va. 20110-2209, on Nov. 21, 2007, and was assigned the ATCC Deposit Number PTA-8766.

### Example 2

### Identification of TrkB Agonists

TrkB agonists (such as antibodies) may be identified using art-recognized methods, including one or more of the following methods. For example, the kinase receptor activation (KIRA) assay described in U.S. Pat. Nos. 5,766,863 and 5,891,650 may be used. This ELISA-type assay is suitable for qualitative or quantitative measurement of kinase activation by measuring the autophosphorylation of the kinase domain of a receptor protein tyrosine kinase (rPTK, e.g. Trk receptor), as well as for identification and characterization of potential agonist or antagonists of a selected rPTK. The first stage of the assay involves phosphorylation of the kinase domain of a kinase receptor, in the present case a TrkB receptor, wherein the receptor is present in the cell membrane of a eukaryotic cell. The receptor may be an endogenous receptor or nucleic acid encoding the receptor, or a receptor construct, may be transformed into the cell. Typically, a first solid phase (e.g., a well of a first assay plate) is coated with a substantially homogeneous population of such cells (usually a mammalian cell line) so that the cells adhere to the solid phase. Often, the cells are adherent and thereby adhere naturally to the first solid phase. If a “receptor construct” is used, it usually comprises a fusion of a kinase receptor and a flag polypeptide. The flag polypeptide is recognized by the capture agent, often a capture antibody, in the ELISA part of the assay. An analyte, such as a candidate agonist, is then added to the wells having the adherent cells, such that the tyrosine kinase receptor (e.g. TrkB receptor) is exposed to (or contacted with) the analyte. This assay enables identification of agonist ligands for the tyrosine kinase receptor of interest (e.g. TrkB). Following exposure to the analyte, the adhering calls are solubilized using a lysis buffer (which has a solubilizing detergent therein) and gentle agitation, thereby releasing cell lysate which can be subjected to the ELISA part of the assay directly, without the need for concentration or clarification of the cell lysate.

The cell lysate thus prepared is then ready to be subjected to the ELISA stage of the assay. As a first step in the ELISA stage, a second solid phase (usually a well of an ELISA microtiter plate) is coated with a capture agent (often a capture antibody) that binds specifically to the tyrosine kinase receptor, or, in the case of a receptor construct, to the flag polypeptide. Coating of the second solid phase is carried out so that the capture agent adheres to the second solid phase. The capture agent is generally a monoclonal antibody, but, as is described in the examples herein, polyclonal antibodies or other agents may also be used. The cell lysate obtained is then exposed to, or contacted with, the adhering capture agent so that the receptor or receptor construct adheres to (or is captured in) the second solid phase. A washing step is then carried out, so as to remove unbound cell lysate, leaving the captured receptor or receptor construct. The adhering or captured receptor or receptor construct is then exposed to, or contacted with, an anti-phosphotyrosine antibody which identifies phosphorylated tyrosine residues in the tyrosine kinase receptor. In the preferred embodiment, the anti-phosphotyrosine antibody is conjugated (directly or indirectly) to an enzyme which catalyses a color change of a non-radioactive color reagent. Accordingly, phosphorylation of the receptor can be measured by a subsequent color change of the reagent. The enzyme can be bound to the anti-phosphotyrosine antibody directly, or a conjugating molecule (e.g., biotin) can be conjugated to the anti-phosphotyrosine antibody and the enzyme can be subsequently bound to the anti-phosphotyrosine antibody via the conjugating molecule. Finally, binding of the anti-phosphotyrosine antibody to the captured receptor or receptor construct is measured, e.g., by a color change in the color reagent.

Following initial identification, the agonist activity of a candidate (e.g., an anti-TrkB monoclonal antibody) can be further confirmed and refined by bioassays, known to test the targeted biological activities. For example, the ability of a candidate to agonize TrkB can be tested in the PC12 neurite outgrowth assay using PC12 cells transfected with full-length TrkB (Jian et al., Cell Signal. 8:365-70, 1996). This assay measures the outgrowth of neurite processes by rat pheocytochroma cells (PC12) in response to stimulation by appropriate ligands. These cells express endogenous TrkA and are therefore responsive to NGF. However, they do not express endogenous TrkB and are therefore transfected with TrkB expression construct in order to elicit response to TrkB agonists. After incubating the transfected cells with the candidate, neurite outgrowth is measured, and e.g., cells with neurites exceeding 2 times the diameter of the cell are counted. Candidates (such as anti-TrkB antibodies) that stimulate neurite outgrowth in transfected PC12 cells demonstrate TrkB agonist activity.

The activation of TrkB may also be determined by using various specific neurons at specific stages of embryonic development. Appropriately selected neurons can be dependent on TrkB activation for survival, and so it is possible to determine the activation of TrkB by following the survival of these neurons in vitro. Addition of candidates to primary cultures of appropriate neurons will lead to survival of these neurons for a period of at least several days if the candidates activate TrkB. This allows the determination of the ability of the candidate (such as an anti-TrkB antibody) to activate TrkB. In one example of this type of assay, the Nodose ganglion from an E15 mouse embryo is dissected, dissociated and the resultant neurons are plated in a tissue culture dish at low density. The candidate antibodies are then added to the media and the plates incubated for 24-48 hours. After this time, survival of the neurons is assessed by any of a variety of methods. Samples which received an agonist will typically display an increased survival rate over samples which receive a control antibody, and this allows the determination of the presence of an agonist. See, e.g., Buchman et al (1993) Development 118(3):989-1001.

TrkB agonist may be identified by their ability to activate downstream signaling in a variety of cell types that express TrkB, either naturally or after transfection of DNA encoding TrkB. This TrkB may be human or other mammalian (such a rodent or primate) TrkB. The downstream signaling cascade may be detected by changes to a variety of biochemical or physiological parameters of the TrkB expressing cell, such as the level of protein expression or of protein phosphorylation of proteins or changes to the metabolic or growth state of the cell (including neuronal survival and/or neurite outgrowth, as described herein). Methods of detecting relevant biochemical or physiological parameters are known in the art.

### Example 3

### Determining Antibody Binding Affinity

Determining binding affinity of antibodies to TrkB may be performed by measuring the binding affinity of monofunctional Fab fragments of the antibody. To obtain monofunctional Fab fragments, an antibody (for example, IgG) can be cleaved with papain or expressed recombinantly. The affinity of an anti-TrkB Fab fragment of an antibody can be determined by surface plasmon resonance (BIAcore3000™ surface plasmon resonance (SPR) system, BIAcore, INC, Piscaway N.J.). CM5 chips can be activated with N-ethyl-N′-(3-dimethylaminopropyl)-carbodiinide hydrochloride (EDC) and N-hydroxysuccinimide (NHS) according to the supplier's instructions. Human TrkB-Fc fusion protein (“hTrkB”) (or any other TrkB, such as rat TrkB) can be diluted into 10 mM sodium acetate pH 5.0 and injected over the activated chip at a concentration of 0.0005 mg/ml. Using variable flow time across the individual chip channels, two ranges of antigen density can be achieved: 200-400 response units (RU) for detailed kinetic studies and 500-1000 RU for screening assays. The chip can be blocked with ethanolamine. Regeneration studies have shown that a mixture of Pierce elution buffer (Product No. 21004, Pierce Biotechnology, Rockford, Ill.) and 4 M NaCl (2:1) effectively removes the bound Fab while keeping the activity of hTrkB on the chip for over 200 injections. HBS-EP buffer (0.01M HEPES, pH 7.4, 0.15 NaCl, 3 mM EDTA, 0.005% Surfactant P29) is used as running buffer for the BIAcore assays. Serial dilutions (0.1-10× estimated K_{D}) of purified Fab samples are injected for 1 min at 100 μl/min and dissociation times of up to 2 h are allowed. The concentrations of the Fab proteins are determined by ELISA and/or SDS-PAGE electrophoresis using a Fab of known concentration (as determined by amino acid analysis) as a standard. Kinetic association rates (kₒₙ) and dissociation rates (k_{off}) (generally measured at 25° C.) are obtained simultaneously by fitting the data to a 1:1 Langmuir binding model (Karlsson, R. Roos, H. Fagerstam, L. Petersson, B. (1994). Methods Enzymology 6:99-110) using the BIAevaluation program. Equilibrium dissociation constant (K_{D}) values are calculated as k_{off}/kₒₙ.

### Example 4

### Kinetic Analysis of Ligand/Receptor Interactions, as Measured by Biacore

General Method:

Interaction analysis was performed at 25° C. using a Biacore 3000 instrument equipped with a CM5 sensor chip (Biacore AB, Uppsala, Sweden). HBS-EP running buffer (used for immobilizing receptors) was purchased from Biacore, along with amine-coupling reagents (NHS, EDC, acetate pH 4.5, and ethanolamine). Fc-fused recombinant human receptors (TrkA, TrkB, TrkC, and P75) and native ligands (NGF, BDNF, NT4/5, and NT3) were either purchased from R&D systems or prepared in-house.

Immobilization of Receptors:

Receptors (3 per chip) were immobilized onto CM5 sensor chips at low levels (typically 500 RU, or 4 fmol/mm²), leaving one flow cell unmodified (per chip) to serve as a reference surface. A standard amine-coupling protocol was used in HBS-EP running buffer at 5 μl/min and involved three steps. Briefly, this involved three steps. First, flow cells were activated with a 7-minute injection of a freshly prepared mixture of 200 mM EDC in 50 mM NHS; this step converted the chip's carboxylic acid groups to reactive esters. Next, receptors were diluted to ˜10 μg/mL in 10 mM sodium acetate buffer at pH 4.5 and coupled to the chip until the desired level had been reached. Finally, excess reactive esters were blocked with a 7-minute injection of 1M sodium ethanolamine-HCl (pH 8.5).

Analysis of Ligands:

Ligands (NGF, BDNF, NT4/5, and NT3) were diluted serially in running buffer (10 mM Hepes (pH 7.4), 150 mM (NH4)₂SO4, 1.5 mM CaCl₂, 1 mM EGTA, and 0.005% (v/v) Tween-20) to concentrations spanning 0.08-250 nM in 5-fold increments. The Fab fragment of antibody 200.38B8 (38B8) was diluted from 0.7-480 nM in 3-fold increments. Samples were injected for 30 seconds at 100 μl/min allowing dissociation times of up to 10 mins. After each binding cycle, receptor surfaces were regenerated with a mildly acidic cocktail (10 mM glycine-HCl (pH 4.0), 500 mM NaCl, and 20 mM EDTA). Duplicate injections confirmed that the assay was reproducible. Kinetic rate constants (kon and koff) were determined by fitting the binding data globally to a mass transport model and binding affinities were calculated from their ratio, KD=koff/kon.

Results:

Most ligand/receptor interactions in the panel were characterized by fast, diffusion-limited on rates, which were beyond the resolution of Biacore (kon˜1×10⁷ 1/Ms). A notable exception was ProNGF, which typically showed slow on rates. Off rates were more variable, for example, interactions of ProNGF or mature NGF with TrkA had very slow off rates, (T1/2>1 hour), whereas the NT3/TrkA interaction decayed within seconds (T1/2=9 sec). The 38B8-Fab/TrkB interaction had a biphasic off rate, so only the initial phase of decay was fit to the model. Receptors were coated at low levels on the chip in order to space them far enough apart (on average) such that dimeric ligands could not cis-bridge inter-molecularly between adjacent receptor molecules. It is unlikely that a dimeric ligand could bridge intra-molecularly between two arms of an Fc-fused receptor molecule, due to steric constraints. By promoting conditions where ligands were forced to bind via only one of their two available binding sites, we minimized avidity issues and feel justified in fitting our data to a simple 1:1 binding model.

TABLE 2
Global kinetic analysis (interactions are ranked from
high to low affinity)

| Ligand/Receptor | kₒₙ (1/Ms) | k_{off} (1/s) | KD (nM) |
|---|---|---|---|
| NGF/TrkA | >1e7 | (1.88 ± 0.02)e−4 | <0.018 ± 0.009 |
| ProNGF/TrkA | 7.16e5 | 1.41e−5 | 0.02 |
| BDNF/TrkB | >1e7 | 1.3e−3 | <0.034 |
| NT45/TrkB | >1e7 | (2.0 ± 0.2)e−3 | <0.20 ± 0.02 |
| NT3/TrkC | >1e7 | 0.0151 | <0.9 |
| NGF/P75 | >1e7 | (4.9 ± 0.5)e−2 | <1.7 ± 0.2 |
| NT3/P75 | >1e7 | 0.164 | <1.7 |
| BDNF/P75 | 8.38e6 | 0.0174 | <2.1 |
| NT3/TrkB | >1e7 | (4.4 ± 0.8)e−2 | <4.4 ± 0.8 |
| NT45/TrkA | (2.1 ± 0.6)e6 | (4 ± 2)e−2 | 17 ± 11 |
| NT3/TrkA | (4.4 ± 1.0)e6 | (8 ± 2)e−2 | 18 ± 6 |
| 38B8-Fab/TrkB | (1.4 ± 0.9)e5 | (6 ± 3)e−3 | 46 ± 10 |
| NT45/P75 | 3.45e6 | 0.305 | 88 |
| ProNGF/P75 | 6.58e5 | 0.0858 | 130 |
| ProNGF/TrkB | — | — | NB |
| ProNGF/TrkC | — | — | NB |
| NGF/TrkB | — | — | NB |
| NGF/TrkC | — | — | NB |
| BDNF/TrkA | — | — | NB |
| BDNF/TrkC | — | — | NB |
| NT45/TrkC | — | — | NB |
| 38B8-Fab/TrkA | — | — | NB |
| 38B8-Fab/TrkC | — | — | NB |
| 38B8-Fab/P75 | — | — | NB |

Notes:
1. “Mean Average ± StDev” (i.e., standard deviation) are given for those interactions that were analyzed in two independent experiments
2. Onrates in italics show very fast offrates that are at the resolution of the Biacore (>1e7 1/Ms) because they approach the diffusion limit. We use this as a cut-off value, which means that the overall KD can only be quoted as a limit.
3. NB = No Binding was detected for certain ligand/receptor pairings (consistent with the known specificity from the literature).

### Example 5

### Direct Administration of a TrkB Agonist Reduces Morbidity in Animals

In experiments carried out in support of the present invention, direct administration of a TrkB agonist was shown to reduce morbidity and CNS tissue damage in animals with a CNS-specific autoimmune disease (FIGS. 1A and 1B). A recombinant form of the naturally occurring TrkB agonist NT4 was produced as described in U.S. Patent Application Publication 2005/0209148 and administered to C57BL/6 female mice following MOG-induction (day 0).

Animals were administered NT4 (10 mg/kg, n=8), daily, beginning 10 days after MOG-induction (FIG. 1A). Vehicle was administered to control animals (n=8). Animals were scored for morbidity as follows: 0=normal; 1=limp tail; 2=moderate hind-limb weakness; 3=moderately severe hind-limb weakness (animal can still walk with difficulty); 4=severe hind-limb weakness (animal can move their hind-limbs but cannot walk); 5=complete hind limb paralysis; and 6=death. The animals treated with NT4 showed significantly reduced morbidity compared to control animals. This result demonstrated that a TrkB agonist was effective in slowing the progression of chronic EAE.

FIG. 1B shows the morbidity of animals treated daily with NT4 during different period of time following MOG induction. Animals were treated with either control IgG (n=9), NT4 from day 3 to day 9 (n=8), or NT4 from day 9 to day 15 (n=8). Both administration schedules were effective in reducing animal morbidity. The protection afforded by NT4 when administered during the earlier time period (i.e., the early “pulse treatment’) is at least as good (if not better) than when administered during the later time period. These results indicate that treatment with the TrkB agonist does not have to continue to the time of symptom onset in order for treatment to be effective. TrkB activation may be targeting steps relatively upstream in the induction of EAE.

### Example 6

### Identification of Highly Selective TrkB Agonist Antibodies

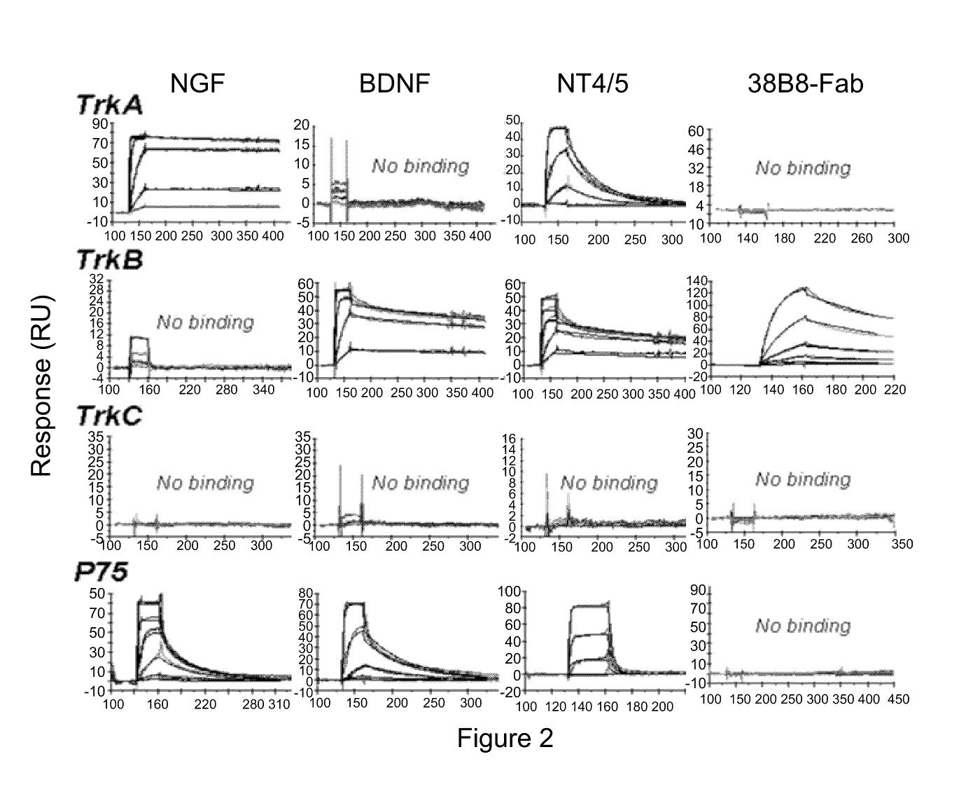

The experiments and observations leading to the identification of TrkB agonist antibodies are described in Examples 1-4. The further characterization of one of the antibodies, antibody 38B8, revealed that the antibody was highly selective for TrkB, while showing nominal binding to TrkA, TrkC, or p75 (FIG. 2). The relative affinities of the 38B8 Fab fragment for TrkA, TrkB, TrkC, and p75 were compared to that of the naturally-occurring agonists NGF, BDNF, and NT4. Each panel in FIG. 2 is a graph showing relative binding versus time. Antibody 38B8 was specific for TrkB and showed no significant binding to the other receptor tyrosine kinases assayed. 38B8 was even more selective for TrkB than the naturally-occurring agonists BDNF and NT4, which showed some cross-reactivity. As expected, NGF demonstrated little binding to TrkB and preferentially bound to TrkA and the p75 neurotrophin receptor. The binding affinity of 38B8 was determined to be 46±10 nM. A kinetic analysis of these ligand-receptor interactions (including Kon, Koff, and Kd data) is provided in Example 4, particularly in Table 2.

BDNF and NT4 appear to have higher affinities for TrkB than the 38B8 antibody agonist. However, the binding experiments were performed with the dimeric forms of the naturally-occurring ligands and a monomeric form of 38B8 Fab. In addition, antibodies generally have much longer circulating half-lives than growth factors allowing them to be effective even while having lower binding affinities for their target receptor. The TrkB agonist antibody was used for further studies.

### Example 7

### A TrkB Agonist Antibody is Effective in Reducing Morbidity in Autoimmune Disease

Animal experiments showed that the TrkB agonist antibody provided significant protection against EAE morbidity compared to a control immunoglobulin (FIG. 3). Following MOG induction, the mice were treated with either 5 mg/kg agonist antibody (38B8, n=9) or a control IgG (n=9), administered on days 9 and 16. The TrkB agonist antibody was also effective when administered as late as 16 days following MOG-induction, and after the onset of clinical symptoms (FIG. 4). Later administration, e.g., 18 days following induction, was less effective in reducing morbidity (FIG. 5A). The difference in clinical symptoms following later treatment was not significant based on two-way ANOVA analysis.

In other animals, the administration of glatiramer acetate (GA, COPAXONE, TYSABRI) 22 days following MOG-induction was effective in reducing morbidity (FIG. 5B). These results suggest that the mechanism of action of the TrkB agonist is different from that of GA, and other current MS drugs.

The TrkB agonist antibody was compared to another current drug for the treatment of MS, dexamethasone. FIG. 6A depicts a graph showing morbidity in animals following the administration of either a TrkB agonist antibody 38B8 (5 mg/kg, weekly on day 9 and day 16) or dexamethasone (4 mg/kg) or ethanol vehicle (control), daily on days 3-12. The results demonstrate that the beneficial effects of the TrkB agonist were similar to that of the immunosuppressant dexamethasone. TrkB agonist-treated animals tended to lose weight compared to control animals (FIG. 6B).

The beneficial effects of the TrkB agonist antibody were dose-dependent. FIGS. 7A and 7B show the difference in animal morbidity following the administration of 1 (n=8) or 5 mg/kg (n=9) 38B8 or 5 (n=10) and 10 mg/kg (n=9) 38B8. The results demonstrated that 5 mg/kg agonist antibody provide more protection from morbidity (i.e., less morbidity) than 1 mg/kg, while 10 mg/kg further reduced morbidity compared to 5 mg/kg. The increase in protection was less pronounced at the higher dosages, suggesting that administering additional TrkB agonist would be of marginal therapeutic value.

### Example 8

### TrkB Agonist Antibodies and NTs Protect Neuronal Cells in Culture

NTs have been shown to promote neuron survival using an in vitro neuron cell survival assay (Davies, A., et al. (1993) Neuron 11565-74). A similar assay was used to demonstrate that TrkB agonist antibodies affect neuronal cells in a manner consistent with naturally-occurring TrkB agonists (see Example 1). Virtually all neurons in a control culture (i.e., without neurotrophic factors) die within 48 hours. However, 60-80% of neurons cultured in the presence of BDNF, NT3, NT4, or NGF survive for 48 hours (Id.).

In experiments performed in support of the invention, increasing amounts of the TrkB agonist antibodies 38B8, 23B8, 36D1, 37D12, and 19H8 (see, e.g., Example 1, including Table 1), were added to neuron cultures as previously described (FIG. 8). With some variation, adding increasing amounts of the TrkB agonist antibodies resulted in increased neuron survival (up to over 75% in the 10-100 pM range for 38B8 and up to over 100% in the 0.1-10 pM range for 19H8). The EC₅₀ value of antibody 38B8 in the neuron survival assay was 0.2 pM. The EC₅₀ values for 38B8, 23B8, 36D1, and 37D12 in the neuron survival assay, as well as the EC₅₀ values for these antibodies in the human KIRA assay and their effect on animal body weight (a recognized response to TrkB agonists), are discussed in Example 1 and summarized in Table 1. Note that antibody 23B8, with a higher EC₅₀ value of 11 pM, failed to produce an effect on animal body weight, suggesting that an EC₅₀ value of below 11 pM is required for TrkB agonist biological activity. Antibody 36D1, with an EC₅₀ value of 5 pM, produced an effect on animal body weight. These data are consistent with results reported for NTs, demonstrating that the TrkB agonist antibody affects neuronal cells in manner consistent with naturally-occurring TrkB agonists.

### Example 9

### TrkB Agonists do not Function Primarily Through Immunosuppression

Current drugs for the treatment of MS (including GA and dexamethasone) are immunomodulators, which exhibit immunosuppressive and other effects with respect to immune response. To determine if the therapeutic effect of TrkB agonists was also at the level of immune suppression, animals were treated with the TrkB agonist antibody (38B8) or vehicle only (control), and the levels of circulating MOG (myelin)-specific antibodies of different isotypes (i.e., IgG1, IgG2a, IgG2b, IgG3, and IgM) were measured (FIG. 9). Although some variation in MOG-specific antibody levels was observed following TrkB agonist treatment, it did not appear to be sufficient to explain the marked reduction in animal morbidity. While not ascribing a particular mechanism to the present invention, the results suggest that TrkB agonists do not function by suppressing the production of anti-MOG antibodies and, therefore, do not appear to function as conventional immunosuppressants.

To determine if TrkB agonists inhibit the ability of T-cells and B-cells to be stimulated by myelin, a splenocyte proliferation assay was used to measure the ability of MOG to stimulate isolated spleen cells in the presence of a TrkB agonist. For comparison, splenocytes were stimulated with MOG in the presence of the immunosuppressant dexamethasone.

Spleen cells from MOG-induced untreated (control) animals (n=9) or MOG-induced TrkB agonist antibody (38B8)-treated animals (n=8) were cultured in vitro in the presence of either MOG alone (0), MOG in combination with the TrkB agonist antibody (38B8; 50 μl/mg), or dexamethasone at one of two different concentrations (10⁻⁸ M and 10⁻⁵ M). Referring to FIG. 10, the various amounts of splenocyte stimulation relative to splenocytes that were not stimulated in vitro with MOG are indicated on the y-axis.

The presence of the TrkB agonist antibody had no apparent effect on splenocyte stimulation, as was also the case with the lower concentration of the immunosuppressant dexamethasone. Dexamethasone interfered with MOG-stimulation at the higher concentration of 10⁻⁵ M.

Overall, splenocytes obtained from TrkB agonist-treated animals respond to MOG stimulation in a manner consistent with control-treated animals, indicating that animals treated with the TrkB agonist retain the ability to produce normal anti-MOG T-cell and/or B-cell responses. Additionally, splenocytes obtained from TrkB agonist-treated animals and control animals respond similarly to the presence of the TrkB agonist in the culture medium. These observations further suggest that mechanisms of action of the immunosuppressant dexamethasone and TrkB agonists are different, and that the mechanism of action of TrkB agonists is not primarily at the level of leukocyte proliferation.

### Example 10

### TrkB Agonists Block Inflammatory Cell Invasion of the CNS and Reduce Demyelination

Histological analysis was performed to better understand the mechanism by which TrkB agonists mediate protection in animals. Spinal cord sections were prepared from control and TrkB agonist antibody (38B8)-treated animals, and then stained with Luxol fast blue to stain myelin, and Cresyl violet to stain cell bodies (FIG. 11). In control animals, the staining showed a region of leukocyte invasion and cell destruction against a background of myelin-expressing neuronal cells. Reduced leukocyte invasion was observed in sections prepared from animals treated with the TrkB agonist. Some TrkB agonist-treated animals showed virtually no evidence of CNS leukocyte invasion.

Identification of the invading cells was performed using antibodies specific for two leukocyte markers, CD3 present on T-cells and CD68 present on macrophages. FIG. 12 shows the results of staining with the CD3 antibody. Numerous brightly staining punctuate regions are apparent, particularly in the same areas that stained darkly with Cresyl violet. The spinal cord section obtained from TrkB agonist-treated mice showed significantly less staining, indicating that T-cell invasion was substantially reduced in treated animals. Spinal cord sections were also stained with the antibody specific for the CD68 marker associated with monocytes (FIG. 13). The sections prepared from control mice stained more strongly than the sections from TrkB agonist-treated mice, particularly in the same regions that stained strongly with Cresyl violet and the CD3-specific antibody. Such observations indicated that spinal cord tissues from TrkB agonist-treated animals show substantially reduced T-cell and monocyte invasion when compared to control animals.

The spinal cord sections were stained with Luxol Fast Blue to measure demyelination. The brains of control mice showed regions of severe demyelination (i.e., the absence of staining, as indicated by the black arrows in FIG. 14) corresponding to areas with heavy lymphocyte invasion, which were not apparent in mice treated with the TrkB agonist. Regions of severe demyelination and/or neuron death were not observed in sections from TrkB agonist-treated animals. Taken together, the results of the histochemical staining of CNS tissue sections demonstrated that TrkB agonist treatment reduces lymphocyte and monocyte invasion of the CNS.

Both naturally-occurring and artificial TrkB agonists are effective in slowing EAE progression. The naturally-occurring agonist NT4 and an agonist antibody were both effective in slowing disease progression. The agonist antibody demonstrated the greatest selectivity for TrkB and was used to further investigate the mechanism by which TrkB agonists affect EAE progression. TrkB agonists were effective when administered before and several days after the onset of EAE symptom (usually day 12 or 13 for these particular animals). Administration up to 16 days following MOG-induction (or about 4 days following the onset of symptoms) was effective in reducing morbidity. The beneficial effects of TrkB agonists are dosage-dependent, with reduced morbidity being associated with increasing dosages of TrkB agonists. Histochemical experiments showed that TrkB agonists reduce invasion of CNS tissues by T-cells and monocytes. Staining for myelin showed reduced damage to nerve cells in TrkB agonist-treated animals.

Unlike the other drugs tested in the assays, TrkB agonists do not function primarily through immunosuppression. This was evidenced in the observation that TrkB agonists did not affect the production of MOG-specific autoimmune antibodies. In addition, splenocytes isolated from MOG-induced animals that were treated with TrkB agonists retained the ability to be stimulated by MOG in vitro. Thus TrkB agonists function in a manner different from that of other compounds, such as GA and dexamethasone.

It is understood that the examples and embodiments described herein are for illustrative purposes only and that various modifications or changes in light thereof will be suggested to persons skilled in the art and are to be included within the spirit and purview of this application. All publications, patents and patent applications cited herein are hereby incorporated by reference in their entirety for all purposes to the same extent as if each individual publication, patent or patent application were specifically and individually indicated to be so incorporated by reference.

We claim:

1. The use of a TrkB agonist in the manufacture of a medicament used for treating an autoimmune disorder affecting the central nervous system in a mammal.

2. The use of claim 1, wherein the autoimmune disorder is experimental autoimmune encephalomyelitis.

3. The use of claim 1, wherein the autoimmune disorder is multiple sclerosis.

4. The use of claim 1, wherein the TrkB agonist is a naturally-occurring TrkB agonist.

5. The use of claim 4, wherein the naturally-occurring TrkB agonist is NT4.

6. The use of claim 4, wherein the naturally-occurring TrkB agonist is BDNF.

7. The use of claim 1, wherein the TrkB agonist is an antibody.

8. The use of claim 7, wherein the TrkB agonist is an antibody fragment or derivative thereof.

9. An isolated monoclonal TrkB agonist antibody produced by the hybridoma strain deposited under ATCC Deposit Number PTA-8766.

10. The hybridoma strain deposited under ATCC Deposit Number PTA-8766.
